(19) United States
(12) Patent Application Publication
(10) Pub. No.: US 2019/0224200 A1
(43) Pub. Date: Jul. 25, 2019

(54) COMBINATIONS FOR THE TREATMENT OF CANCER

(71) Applicant: IGNYTA, INC., San Diego, CA (US)

(72) Inventors: Eric Santos MARTIN, San Diego, CA (US); Yumi YOKOYAMA, San Diego, CA (US)

(73) Assignee: IGNYTA, INC., San Diego, CA (US)

(21) Appl. No.: 16/327,301

(22) PCT Filed: Aug. 22, 2017
(86) PCT No.: PCT/US2017/048075

§ 371 Date: Feb. 21, 2019

Related U.S. Application Data
(60) Provisional application No. 62/521,176, filed on Jun. 16, 2017.
(60) Provisional application No. 62/379,169, filed on Aug. 24, 2016.

(51) Int. Cl. A61K 31/513; A61K 9/00; A61P 35/00; A61K 39/395

(52) U.S. Cl. CPC A61K 31/513; A61K 9/0053; A61K 2039/505; A61P 35/00; A61K 39/39558; A61K 9/0019

(57) ABSTRACT

The present disclosure relates to certain combinations for the treatment of cancer in a subject, comprising one or more inhibitors of Tyro3, Axl, Mer, or c-Met, together with one or more compounds that are inhibitors of programmed cell death protein 1 (PD-1) or programmed death-ligand 1 (PD-L1).

### CROSS-REFERENCE TO RELATED APPLICATIONS

This application claims the priority benefit of U.S. provisional application Nos. 62/379,169, filed on Aug. 24, 2016, and 62/521,176, filed on Jun. 16, 2017, both of which are incorporated by reference herein in their entirety.

### FIELD

The present disclosure relates to certain combinations for the treatment of cancer in a subject, comprising one or more inhibitors of Tyro3, Axl, Mer, or c-Met, together with one or more anti-PD-1 agents or anti-PD-L1 agents. The present disclosure also provides methods for the treatment of a subject in need thereof, comprising administering to the subject a combination of comprising one or more inhibitors of Tyro3, Axl, Mer, or c-Met, together with one or more anti-PD-1 agents or anti-PD-L1 agents. The present disclosure also relates to treating cancer in a subject utilizing pharmaceutical compositions comprising a combination of comprising one or more inhibitors of Tyro3, Axl, Mer, or c-Met, together with one or more anti-PD-1 agents or anti-PD-L1 agents.

### BACKGROUND

Programmed cell death protein 1 (PD-1, also known as CD279) is a 50-55 kDa type I transmembrane receptor. PD-1 is expressed on activated T, B, and myeloid lineage cells, and plays an important role in down-regulating the immune system by preventing the activation of T cells. Two ligands for PD-1 have been identified, PD ligand 1 (PD-L1, also known as CD274) and ligand 2 (PD-L2, also known as CD273), both of which belong to the B7 superfamily. PD-L1 is expressed on many cell types, including T, B, endothelial and epithelial cells, and antigen-presenting cells. However, PD-L2 is narrowly expressed on professional antigen-presenting cells, such as dendritic cells and macrophages.

The receptors Tyro3, Axl, and Mer, collectively “TAM,” comprise a unique family of receptor tyrosine kinases, in that as a group they play no essential role in embryonic development. Instead, they function as homeostatic regulators in adult tissues and organ systems that are subject to continuous challenge and renewal throughout life. Their regulatory roles are prominent in the mature immune, reproductive, hematopoietic, vascular, and nervous systems. The TAMs and their ligands—Gas6 and Protein S—are essential for the efficient phagocytosis of apoptotic cells and membranes in these tissues; and in the immune system, they act as pleiotropic inhibitors of the innate inflammatory response to pathogens. Deficiencies in TAM signaling are thought to contribute to chronic inflammatory and autoimmune disease in humans, and aberrantly elevated TAM signaling is strongly associated with cancer progression, metastasis, and resistance to targeted therapies. Inhibitors of TAM have shown promise in the treatment of subjects having cancer.

Herein are described methods of methods of treating cancer in a subject comprising administering to said subject a therapeutically effective amount of a combination comprising (a) an inhibitor of Tyro3, Axl, Mer, or c-Met, and (b) an anti-PD-1 agent or an anti-PD-L1 agent. Also described herein is a medicament for use in treating cancer in a subject, comprising a first composition comprising an inhibitor of Tyro3, Axl, Mer, or c-Met and a second composition comprising an anti-PD-1 agent or an anti-PD-L1 agent. Also described herein is a combination for use in treating a cancer in a subject, said combination comprising (a) an inhibitor of Tyro3, Axl, Mer, or c-Met, and (b) an anti-PD-1 agent or an anti-PD-L1 agent. Also described herein is a combination for use in treating a cancer in a subject, said combination comprising (a) a first pharmaceutical composition comprising an inhibitor of Tyro3, Axl, Mer, or c-Met, and (b) a second pharmaceutical composition comprising an anti-PD-1 agent or an anti-PD-L1 agent.

### SUMMARY

In an embodiment is provided a method of treating cancer in a subject, comprising administering to said subject a therapeutically effective amount of a combination comprising (a) an inhibitor of Tyro3, Axl, Mer, or c-Met, and (b) an anti-PD-1 agent or an anti-PD-L1 agent.

In an embodiment is provided a method of treating, ameliorating the symptoms of, delaying the onset of or delaying the progression of cancer in a subject comprising the steps of:
(a) determining whether modulation of Tyro3, Axl, Mer, or c-Met activity is defective in a cell population of said subject, and if said modulation of Tyro3, Axl, Mer, or c-Met activity is defective,
(b) administering a combination to said subject comprising (i) an inhibitor of Tyro3, Axl, Mer, or c-Met, and (ii) an anti-PD-1 agent or an anti-PD-L1 agent thereby treating, ameliorating the symptoms of, delaying the onset of or delaying the progression of cancer.

In an embodiment is provided a method of treating cancer in a subject, comprising administering to said subject a therapeutically effective amount of a combination comprising (a) an inhibitor of Tyro3, Axl, Mer, or c-Met, and (b) an anti-PD-1 agent or an anti-PD-L1 agent, wherein one or more cancer cells in said subject is determined to possess at least one molecular alteration in one or more of Tyro3, Axl, Mer, or c-Met prior to administration of said inhibitor of Tyro3, Axl, Mer, or c-Met.

In an embodiment is provided a method of treating cancer in a subject, wherein one or more cancer cells in said subject is determined to possess at least one molecular alteration in one or more of Tyro3, Axl, Mer, or c-Met, comprising administering to said subject a therapeutically effective amount of a combination comprising (a) an inhibitor of Tyro3, Axl, Mer, or c-Met, and (b) an anti-PD-1 agent or an anti-PD-L1 agent.

### BRIEF DESCRIPTION OF THE DRAWINGS

FIG. 1 shows in vivo efficacy of an inhibitor of Tyro3, Axl, Mer, or c-Met alone, or in combination with an anti-PD-1 agent in inhibiting growth of colon carcinoma in CT26 syngeneic mouse model. In the figure legend, “RXDX-106” refers to compound N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide; “α-PD-1 Ab” refers to anti-PD-1 antibody; “p.o.” refers to administration per os or by oral gavage; “q.d.” refers to once daily; “2×/week” refers to twice every week.

FIG. 2 shows in vivo efficacy of an inhibitor of Tyro3, Axl, Mer, or c-Met alone, or in combination with an anti-PD-1 agent in triggering release of IFN-γ in treated mice. In the figure legend, “RXDX-106” refers to compound N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide; “anti-PD-1 Ab” refers to anti-PD-1 antibody.

### DETAILED DESCRIPTION

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

As used herein, the term “N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide” means the compound having Chemical Abstracts Registry No. 1437321-24-8, and having the chemical structure:

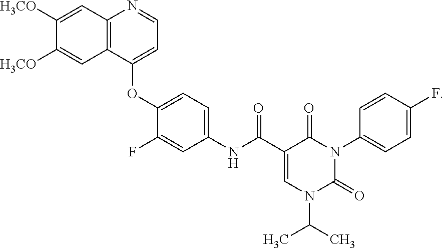 [SMILES: COc1cc2nccc(Oc3ccc(NC(=O)c4cn(C(C)C)c(=O)n(-c5ccc(F)cc5)c4=O)cc3F)c2cc1OC]

The preparation of N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, and pharmaceutically acceptable salts thereof, is described in U.S. Pat. No. 9,029,538, the disclosure of which is incorporated herein by reference in its entirety.

As used herein, the term “about” means either within plus or minus 10% of the provided value, or rounded to the nearest significant figure, in all cases inclusive of the provided value. Where ranges are provided, they are inclusive of the boundary values.

As used herein, the terms “administration” and “administering” mean the delivery of a bioactive composition or formulation by an administration route including, but not limited to, intravenous, intra-arterial, intramuscular, intraperitoneal, subcutaneous, intramuscular, topically, or combinations thereof.

As used herein, the term “anti-PD-1 agent” means an agent capable binding to PD-1 and blocking the interaction of PD-1 with its ligands, such as PD-L1 and PD-L2. In an embodiment, the anti-PD-1 agent is a small molecule. In an embodiment, the anti-PD-1 agent is an antibody. In an embodiment, the anti-PD-1 agent is a monoclonal antibody. In an embodiment, the anti-PD-1 agent is a humanized antibody. In an embodiment, the anti-PD-1 agent is a humanized, monoclonal antibody. In an embodiment, the anti-PD-1 agent is a fully human antibody. In an embodiment, the anti-PD-1 agent is a fully human, monoclonal antibody. In an embodiment, the anti-PD-1 agent is nivolumab. In an embodiment, the anti-PD-1 agent is pembrolizumab.

As used herein, the term “anti-PD-L1 agent” means an agent capable binding to PD-L1 and blocking the interaction of PD-L1 with its receptor PD-1. In an embodiment, the anti-PD-L1 agent is a small molecule. In an embodiment, the anti-PD-L1 agent is an antibody. In an embodiment, the anti-PD-L1 agent is a monoclonal antibody. In an embodiment, the anti-PD-L1 agent is a humanized antibody. In an embodiment, the anti-PD-L1 agent is a humanized, monoclonal antibody. In an embodiment, the anti-PD-L1 agent is a fully human antibody. In an embodiment, the anti-PD-L1 agent is a fully human, monoclonal antibody. In an embodiment, the anti-PD-L1 agent is atezolizumab. In an embodiment, the anti-PD-L1 agent is avelumab. In an embodiment, the anti-PD-L1 agent is durvalumab

As used herein, the term “antibody” means an immunoglobulin that specifically binds to, and is thereby defined as complementary with, a particular spatial and polar organization of another molecule. The antibody can be monoclonal or polyclonal and can be prepared by techniques that are well known in the art, such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular target is maintained.

As used herein, the term “biological sample,” means a sample obtained from an organism that may be used in a diagnostic or monitoring assay. The sample may be of a healthy tissue, diseased tissue or tissue suspected of being diseased tissue. The sample may be a biopsy taken, for example, during a surgical procedure. The sample may be collected via means of fine needle aspiration, scraping or washing a cavity to collect cells or tissue therefrom. The sample may be of a tumor such as, for example, solid and hematopoietic tumors as well as of neighboring healthy tissue. The sample may be a smear of individual cells or a tissue section. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses clinical samples, and also includes cells in cell culture, cell supernatants, cell lysates, cell extracts, cell homogenates, subcellular components including synthesized proteins, serum, plasma, bodily and other biological fluids, and tissue samples. The biological sample can contain compounds that are not naturally intermixed with the cell or tissue in nature such as preservatives, anticoagulants, buffers, fixatives, nutrients, antibiotics or the like. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample.

As used herein, the term “biomarker” means one or more compounds whose level of nucleic acid or protein product has a quantitatively differential concentration or level with respect to an aspect of a biological state of a subject. The term “biomarker” may be used herein interchangeably with the term “marker.” The level of the biomarker can be measured at both the nucleic acid level as well as the polypeptide level. At the nucleic acid level, a nucleic acid gene or a transcript which is transcribed from any part of the subject's chromosomal and extrachromosomal genome, including for example the mitochondrial genome, may be measured. Preferably an RNA transcript, more preferably an RNA transcript includes a primary transcript, a spliced transcript, an alternatively spliced transcript, or an mRNA of the biomarker is measured. At the polypeptide level, a pre-propeptide, a propeptide, a mature peptide or a secreted peptide of the biomarker may be measured. A biomarker can be used either solely or in conjunction with one or more other identified biomarkers so as to allow correlation to the biological state of interest as defined herein. Specific examples of biomarkers covered by the present disclosure include Tyro3, Axl, Mer, and c-Met.

As used herein, the terms “cancer” or “tumor” may be used interchangeably. These terms mean the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers. In certain embodiments, the cancer is a solid tumor, a soft tissue tumor, or a metastatic lesion. The terms also refer to solid tumors named for the type of cells that form them, cancer of blood, bone marrow, or the lymphatic system. Examples of solid tumors include but are not limited to sarcomas and carcinomas. Examples of cancers of the blood include but are not limited to leukemias, lymphomas and myeloma. The terms include but are not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

As used herein, the term “chemotherapeutic agent”, means a chemical substance, such as a cytotoxic or cytostatic agent, that is used to treat a condition, particularly cancer. In some embodiments, the chemotherapeutic agents include one or more compounds disclosed herein, or a pharmaceutically acceptable salt thereof.

As used herein, the terms “combination” and “in combination with” mean the administration of one or more compounds disclosed herein, or a pharmaceutically acceptable salt thereof, or a combination disclosed herein together with an at least one additional pharmaceutical or medicinal agent (e.g., an anti-cancer agent), either sequentially or simultaneously. It includes dosing simultaneously, or within minutes or hours of each other, or on the same day, or on alternating days, or dosing the compound disclosed herein on a daily basis, or multiple days per week, or weekly basis, for example, while administering another compound such as a chemotherapeutic agent on the same day or alternating days or weeks or on a periodic basis during a time simultaneous therewith or concurrent therewith, or at least a part of the time during which the compound disclosed herein is dosed. For example, one or more compounds disclosed herein, or a pharmaceutically acceptable salt thereof, could be dosed every day or several days a week while the chemotherapeutic agent is dosed on alternating days or alternating weeks or other periods of time, such as every 1, 2, 3, 4, 5, 6, 7, 8, 9, 10, 11. 12, 13, 14 or more days.

As used herein, the term “contact” when used in reference to specificity or specific binding means two molecules are close enough so that short range non-covalent chemical interactions, such as Van der Waal forces, hydrogen bonding, hydrophobic interactions, and the like, dominate the interaction of the molecule.

As used herein, the term “cell line” means to one or more generations of cells which are derived from a clonal cell. The term “clone,” or “clonal cell,” means a single cell which is expanded to produce an isolated population of phenotypically similar cells (i.e., a “clonal cell population”).

As used herein, the term “PD-1” means Programmed Cell Death 1 protein. PD-1 is also referred to by those of ordinary skill in the art as CD279.

As used herein, the term “PD-L1” means Programmed Death-Ligand 1. PD-L1 is also referred to by those of ordinary skill in the art as CD274.

As used herein, the term “immunohistochemistry”, means the process of localizing antigens (e.g., proteins) in biological samples, cells and/or cells of a tissue section exploiting the principle of antibodies binding specifically to antigens. Immunohistochemical staining is widely used in the diagnosis of abnormal cells such as those found in cancerous tumors. Specific molecular markers are characteristic of particular cellular events, such as cell proliferation or cell death. Visualizing an antibody-antigen interaction can be accomplished in a number of ways. In the most common instance, an antibody is conjugated to an enzyme, such as peroxidase, that can catalyze a color-producing reaction. Alternatively, the antibody can also be tagged to a fluorophore thus employing the principles of immunofluorescence. Immunohistochemistry can also be used to evaluate tumor content in the sample on which qPCR is carried out in order to account for the fact that qPCR result will be influenced by the amount of tumor tissue present.

As used herein, the terms “monoclonal antibody,” “mAb” and “MAB” mean an antibody that is an immunoglobulin produced by a single clone of lymphocytes which recognizes only a single epitope on an antigen. For example, a monoclonal antibody useful for the methods disclosed herein displays a single binding specificity and affinity for a particular epitope of one or more tyrosine kinases.

As used herein, the term “one or more molecular alterations” means any variation in the genetic or protein sequence in or more cells of a subject as compared to the corresponding wild-type genes or proteins. One or more molecular alterations include, but are not limited to, genetic mutations, gene amplifications, splice variants, deletions, insertions/deletions, gene rearrangements, single-nucleotide variations (SNVs), insertions, and aberrant RNA/protein expression.

As used herein, the term “pharmaceutically acceptable salt” means those salts that retain the biological effectiveness and properties of the parent compound.

The term “polyclonal antibody” as used herein means a composition of different antibody molecules which is capable of binding to or reacting with several different specific antigenic determinants on the same or on different antigens. The variability in antigen specificity of a polyclonal antibody is located in the variable regions of the individual antibodies constituting the polyclonal antibody, in particular in the complementarity determining regions (CDRs). Preferably, the polyclonal antibody is prepared by immunization of an animal with the target tyrosine kinases or portions thereof. Alternatively, the polyclonal antibody may be prepared by mixing multiple monoclonal antibodies having desired specificity to a target tyrosine kinase.

The term “selectively binds” as used herein means as the situation in which one member of a specific intra- or inter-species binding pair will not show any significant binding to molecules other than its specific intra- or inter-species binding partner (e.g., an affinity of about 100-fold less), which means that only minimal cross-reactivity occurs.

As used herein in reference to the binding of two molecules or one or more compounds and a complex of molecules, the term “specific” means the specific recognition of one for the other and the formation of a stable complex, as compared to substantially less recognition of other molecules and the lack of formation of stable complexes with such other molecules. Preferably, “specific,” in reference to binding, means that to the extent that one or more compounds forms complexes with other molecules or complexes, it forms at least fifty percent of the complexes with the molecule or complex for which it has specificity. Generally, the molecules or complexes have areas on their surfaces or in cavities giving rise to specific recognition between the two binding moieties. Exemplary of specific binding are antibody-antigen interactions, enzyme-substrate interactions, polynucleotide hybridizations and/or formation of duplexes, cellular receptor-ligand interactions, and so forth.

As used herein, the term “therapeutically effective amount” means that amount of the compound, compounds or combination of compounds being administered which will relieve to some extent one or more of the symptoms of the disorder being treated. In reference to the treatment of a cancer, a therapeutically effective amount means that amount which has the effect of (1) reducing the size of a cancer tumor, (2) inhibiting (that is, slowing to some extent, preferably stopping) cancer tumor metastasis, (3) inhibiting to some extent (that is, slowing to some extent, preferably stopping) cancer tumor growth, and/or, (4) relieving to some extent (or, preferably, eliminating) one or more symptoms associated with the cancer.

In an embodiment is provided a method of treating cancer in a subject, comprising administering to said subject a therapeutically effective amount of a combination comprising (a) an inhibitor of Tyro3, Axl, Mer, or c-Met, and (b) an anti-PD-1 agent or an anti-PD-L1 agent.

In an embodiment is provided a method of treating, ameliorating the symptoms of, delaying the onset of or delaying the progression of cancer in a subject comprising the steps of:
(a) determining whether modulation of Tyro3, Axl, Mer, or c-Met activity is defective in a cell population of said subject, and if said modulation of Tyro3, Axl, Mer, or c-Met activity is defective,
(b) administering a combination to said subject comprising (i) an inhibitor of Tyro3, Axl, Mer, or c-Met, and (ii) an anti-PD-1 agent or an anti-PD-L1 agent, thereby treating, ameliorating the symptoms of, delaying the onset of or delaying the progression of cancer.

In an embodiment is provided a method of treating cancer in a subject, comprising administering to said subject a therapeutically effective amount of a combination comprising (a) an inhibitor of Tyro3, Axl, Mer, or c-Met, and (b) an anti-PD-1 agent or an anti-PD-L1 agent, wherein one or more cancer cells in said subject is determined to possess at least one molecular alteration in one or more of Tyro3, Axl, Mer, or c-Met prior to administration of said inhibitor of Tyro3, Axl, Mer, or c-Met.

In an embodiment is provided a method of treating cancer in a subject, wherein one or more cancer cells in said subject is determined to possess at least one molecular alteration in one or more of Tyro3, Axl, Mer, or c-Met, comprising administering to said subject a therapeutically effective amount of a combination comprising (a) an inhibitor of Tyro3, Axl, Mer, or c-Met, and (b) an anti-PD-1 agent or an anti-PD-L1 agent.

In an embodiment is provided any of the methods described herein, wherein said combination comprises an inhibitor of Tyro3 and an anti-PD-1 agent or an anti-PD-L1 agent.

In an embodiment is provided any of the methods described herein, wherein said combination comprises an inhibitor of Axl and an anti-PD-1 agent or an anti-PD-L1 agent.

In an embodiment is provided any of the methods described herein, wherein said combination comprises an inhibitor of Mer and an anti-PD-1 agent or an anti-PD-L1 agent.

In an embodiment is provided any of the methods described herein, wherein said combination comprises an inhibitor of c-Met and an anti-PD-1 agent or an anti-PD-L1 agent.

In an embodiment is provided any of the methods described herein, wherein said inhibitor of Tyro3, Axl, Mer, or c-Met is N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, or a pharmaceutically acceptable salt thereof.

In an embodiment is provided any of the methods described herein, wherein said inhibitor of Tyro3, Axl, and/or Mer is selected from N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, cabozantinib, bosutinib, crizotinib, vandetinib, sunitenib, lesaurtinib, neratinib, AT9283, R406, foretinib, MK-2461, BMS-777607, LY2801653, SU-14813, S49076, BMS-796302, BGB324, amuvatinib (MP-470), JNJ-28312141, GSK2606414, Ki-20227, spiroindoline, UNC569, UNC1062, UNC2025, and LDC1267.

In an embodiment is provided any of the methods described herein, wherein said inhibitor of c-Met is selected from N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, crizotinib, PF-002341066 (Pfizer), cabozantinib, tivantinib, onatuzumab, tepotinib, savolitinib, SAR-125844 (Sanofi), S-49076 (Servier), MGCD-265 (Mirati), merestinib, golvatinib, foretinib, emibetuzumab, capmatinib, BMS-777607 (Bristol-Myers Squibb), AMG-337 (Amgen), TAS-115 (Taiho), ningetinib, metatinib, LY-3164530 (Eli Lilly), JNJ-38877618 (Johnson & Johnson), ABT-700 (Abbott), BPI9016M (Betta Pharmaceuticals), ARGX-111 (arGEN-X), AMG-208 (Amgen), altiratinib, X-379 (Xcovery), STI-A150x (Sorrento Therapeutics), PRS-110 (Pieris), MM-131 (Merrimack), KTN-0216 (Koltan), EN1-mAb (Genmab), boxitinib, ASP-08001 (Ascepion Pharmaceuticals), ASP-08126 (Ascepion Pharmaceuticals), ACMI-0831 (Abion), and ABN-401 (Abion), or a pharmaceutically acceptable salt thereof.

In an embodiment is provided any of the methods described herein, wherein said combinations comprises N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, or a pharmaceutically acceptable salt thereof, and an anti-PD-1 agent or an anti-PD-L1 agent.

In an embodiment is provided any of the methods described herein, wherein said combinations comprises crizotinib, or a pharmaceutically acceptable salt thereof, and an anti-PD-1 agent or an anti-PD-L1 agent.

In an embodiment is provided any of the methods described herein, wherein said anti-PD-1 agent or anti-PD-L1 agent is a monoclonal antibody.

In an embodiment is provided any of the methods described herein, wherein said monoclonal antibody is a fully human monoclonal antibody.

In an embodiment is provided any of the methods described herein, wherein said anti-PD-1 agent or anti-PD-L1 agent is selected from nivolumab, pembrolizumab, atezolizumab, avelumab, and durvalumab.

In an embodiment is provided any of the methods described herein, wherein said anti-PD-1 agent or anti-PD-L1 agent is nivolumab.

In an embodiment is provided any of the methods described herein, wherein said anti-PD-1 agent or anti-PD-L1 agent is pembrolizumab.

In an embodiment is provided any of the methods described herein, wherein said anti-PD-1 agent or anti-PD-L1 agent is atezolizumab.

In an embodiment is provided any of the methods described herein, wherein said anti-PD-1 agent or anti-PD-L1 agent is avelumab.

In an embodiment is provided any of the methods described herein, wherein said anti-PD-1 agent or anti-PD-L1 agent is durvalumab.

In an embodiment is provided any of the methods described herein, wherein said inhibitor of Tyro3, Axl, Mer, or c-Met and said anti-PD-1 agent or anti-PD-L1 agent are administered to said subject simultaneously.

In an embodiment is provided any of the methods described herein, wherein said inhibitor of Tyro3, Axl, Mer, or c-Met and said anti-PD-1 agent or anti-PD-L1 agent are administered to said subject sequentially.

In an embodiment is provided any of the methods described herein, wherein said inhibitor of Tyro3, Axl, Mer, or c-Met is administered to said subject orally.

In an embodiment is provided any of the methods described herein, wherein said anti-PD-1 agent or anti-PD-L1 agent is administered to said subject intravenously.

In an embodiment is provided any of the methods described herein, wherein said inhibitor of Tyro3, Axl, Mer, or c-Met is administered to said subject orally and said anti-PD-1 agent or anti-PD-L1 agent is administered to said subject intravenously.

In an embodiment is provided any of the methods described herein, wherein said anti-PD-1 agent or anti-PD-L1 agent is administered to said subject every 3 weeks.

In an embodiment is provided any of the methods described herein, wherein said anti-PD-1 agent or anti-PD-L1 agent is administered to said subject in four doses every 3 weeks.

In an embodiment is provided any of the methods described herein, wherein said anti-PD-1 agent or anti-PD-L1 agent is administered to said subject in a dose of 2 mg per kilogram of subject weight. In an embodiment is provided any of the methods described herein, wherein said anti-PD-1 agent or anti-PD-L1 agent is administered to said subject in a dose of 3 mg per kilogram of subject weight. In an embodiment is provided any of the methods described herein, wherein said anti-PD-1 agent or anti-PD-L1 agent is administered to said subject in a dose of about 1 mg per kilogram of subject weight, or about 2 mg per kilogram of subject weight, or about 3 mg per kilogram of subject weight, or about 4 mg per kilogram of subject weight, or about 5 mg per kilogram of subject weight, or about 6 mg per kilogram of subject weight, or about 7 mg per kilogram of subject weight, or about 8 mg per kilogram of subject weight, or about 9 mg per kilogram of subject weight, or about 10 mg per kilogram of subject weight, or about 11 mg per kilogram of subject weight, or about 12 mg per kilogram of subject weight, or about 13 mg per kilogram of subject weight, or about 14 mg per kilogram of subject weight, or about 15 mg per kilogram of subject weight, or about 16 mg per kilogram of subject weight, or about 17 mg per kilogram of subject weight, or about 18 mg per kilogram of subject weight, or about 19 mg per kilogram of subject weight, or about 20 mg per kilogram of subject weight, or about 25 mg per kilogram of subject weight, or about 30 mg per kilogram of subject weight, or about 35 mg per kilogram of subject weight, or about 40 mg per kilogram of subject weight, or about 45 mg per kilogram of subject weight, or about 50 mg per kilogram of subject weight, or about 55 mg per kilogram of subject weight, or about 60 mg per kilogram of subject weight, or about 65 mg per kilogram of subject weight, or about 70 mg per kilogram of subject weight, or about 75 mg per kilogram of subject weight, or about 80 mg per kilogram of subject weight, or about 85 mg per kilogram of subject weight, or about 90 mg per kilogram of subject weight, or about 95 mg per kilogram of subject weight, or about 100 mg per kilogram of subject weight, or about 125 mg per kilogram of subject weight, or about 150 mg per kilogram of subject weight, or about 200 mg per kilogram of subject weight, or about 225 mg per kilogram of subject weight, or about 250 mg per kilogram of subject weight, or about 275 mg per kilogram of subject weight, or about 300 mg per kilogram of subject weight, or about 325 mg per kilogram of subject weight, or about 350 mg per kilogram of subject weight, or about 375 mg per kilogram of subject weight, or about 400 mg per kilogram of subject weight, or about 425 mg per kilogram of subject weight, or about 450 mg per kilogram of subject weight, or about 475 mg per kilogram of subject weight, or about 500 mg per kilogram of subject weight. In an embodiment is provided any of the methods described herein, wherein said anti-PD-1 agent or anti-PD-L1 agent is administered to said subject in a dose of 200 mg every 3 weeks.

In an embodiment is provided any of the methods described herein, wherein said anti-PD-1 agent or anti-PD-L1 agent is administered to said subject in a dose of 10 mg per kilogram of subject weight.

In an embodiment is provided any of the methods described herein, wherein said anti-PD-1 agent or anti-PD-L1 agent is administered to said subject in a dose of 10 mg per kilogram of the weight of said subject twice every week.

In an embodiment is provided any of the methods described herein, wherein said anti-PD-1 agent or anti-PD-L1 agent is administered to said subject every 3 weeks.

In an embodiment is provided any of the methods described herein, wherein said anti-PD-1 agent or anti-PD-L1 agent is administered to said subject every 1 week, or every 2 weeks, or every 3 weeks, or every 4 weeks, or every 5 weeks, or every 6 weeks, or every 7 weeks, or every 8 weeks, or every 3 months, or every 4 months, or every 5 months, or every 6 months, or every 7 months, or every 8 months, or every 9 months, or every 10 months, or every 11 months, or every 12 months.

In an embodiment is provided any of the methods described herein, wherein said anti-PD-1 agent or anti-PD-L1 agent is administered to said subject twice every week.

In an embodiment is provided any of the methods described herein, wherein said anti-PD-1 agent or anti-PD-L1 agent is administered to said subject in a dose of 3 mg per kilogram of the weight of said subject every 3 weeks. In an embodiment is provided any of the methods described herein, wherein said anti-PD-1 agent or anti-PD-L1 agent is administered to said subject in a dose of 200 mg every 3 weeks.

In an embodiment is provided any of the methods described herein, wherein said anti-PD-1 agent or anti-PD-L1 agent is administered to said subject in a dose of 10 mg per kilogram of the weight of said subject twice every week.

In an embodiment is provided any of the methods described herein, wherein said anti-PD-1 agent or anti-PD-L1 agent is administered to said subject in a dose of 3 mg per kilogram of the weight of said subject every 3 weeks for a total of 4 doses.

In an embodiment is provided any of the methods described herein, wherein said inhibitor of Tyro3, Axl, Mer, or c-Met is administered to said subject at least once per day. In an embodiment is provided any of the methods described herein, wherein said inhibitor of Tyro3, Axl, Mer, or c-Met is administered to said subject at least once per day, or twice per day, or three times per day, or four times per day, or five times per day, or six times per day, or seven times per day, or eight times per day, or nine times per day, or ten times per day.

In an embodiment is provided any of the methods described herein, wherein said inhibitor of Tyro3, Axl, Mer, or c-Met is administered to said subject in a dose of about 0.1 mg per kilogram of subject weight to about 1000 mg per kilogram of subject weight. In an embodiment is provided any of the methods described herein, wherein said inhibitor of Tyro3, Axl, Mer, or c-Met is administered to said subject in a dose of about 0.1 mg per kilogram of subject weight to about 750 mg per kilogram of subject weight, or from about 0.1 mg per kilogram of subject weight to about 650 mg per kilogram of subject weight, or from about 0.1 mg per kilogram of subject weight to about 575 mg per kilogram of subject weight, or from about 0.1 mg per kilogram of subject weight to about 550 mg per kilogram of subject weight, or from about 0.1 mg per kilogram of subject weight to about 525 mg per kilogram of subject weight, or from about 0.1 mg per kilogram of subject weight to about 500 mg per kilogram of subject weight, or from about 0.1 mg per kilogram of subject weight to about 475 mg per kilogram of subject weight, or from about 0.1 mg per kilogram of subject weight to about 450 mg per kilogram of subject weight, or from about 0.1 mg per kilogram of subject weight to about 425 mg per kilogram of subject weight, or from about 0.1 mg per kilogram of subject weight to about 400 mg per kilogram of subject weight, or from about 0.1 mg per kilogram of subject weight to about 375 mg per kilogram of subject weight, or from about 0.1 mg per kilogram of subject weight to about 350 mg per kilogram of subject weight, or from about 0.1 mg per kilogram of subject weight to about 325 mg per kilogram of subject weight, or from about 0.1 mg per kilogram of subject weight to about 300 mg per kilogram of subject weight, or from about 0.1 mg per kilogram of subject weight to about 275 mg per kilogram of subject weight, or from about 0.1 mg per kilogram of subject weight to about 250 mg per kilogram of subject weight, or from about 0.1 mg per kilogram of subject weight to about 225 mg per kilogram of subject weight, or from about 0.1 mg per kilogram of subject weight to about 200 mg per kilogram of subject weight, or from about 0.1 mg per kilogram of subject weight to about 175 mg per kilogram of subject weight, or from about 0.1 mg per kilogram of subject weight to about 150 mg per kilogram of subject weight, or from about 0.1 mg per kilogram of subject weight to about 125 mg per kilogram of subject weight, or from about 0.1 mg per kilogram of subject weight to about 100 mg per kilogram of subject weight, or from about 0.1 mg per kilogram of subject weight to about 75 mg per kilogram of subject weight, or from about 0.1 mg per kilogram of subject weight to about 50 mg per kilogram of subject weight, or from about 0.1 mg per kilogram of subject weight to about 25 mg per kilogram of subject weight, or from about 0.1 mg per kilogram of subject weight to about 20 mg per kilogram of subject weight, or from about 0.1 mg per kilogram of subject weight to about 15 mg per kilogram of subject weight, or from about 0.1 mg per kilogram of subject weight to about 10 mg per kilogram of subject weight, or from about 0.1 mg per kilogram of subject weight to about 5 mg per kilogram of subject weight, or from about 0.1 mg per kilogram of subject weight to about 2.5 mg per kilogram of subject weight, or from about 0.1 mg per kilogram of subject weight to about 2 mg per kilogram of subject weight, or from about 0.1 mg per kilogram of subject weight to about 1 mg per kilogram of subject weight.

In an embodiment is provided any of the methods described herein, wherein said inhibitor of Tyro3, Axl, Mer, or c-Met is administered to said subject in a dose of about from about 0.1 mg per kilogram of subject weight to about 30 mg per kilogram of subject weight.

In an embodiment is provided any of the methods described herein, wherein said inhibitor of Tyro3, Axl, Mer, or c-Met is administered to said subject in a dose of about 1 mg, or about 5 mg, or about 10 mg, or about 15 mg, or about 20 mg, or about 25 mg, or about 30 mg, or about 35 mg, or about 40 mg, or about 45 mg, or about 50 mg, or about 55 mg, or about 60 mg, or about 65 mg, or about 70 mg, or about 75 mg, or about 80 mg, or about 85 mg, or about 90 mg, or about 95 mg, or about 100 mg, or about 125 mg, or about 150 mg, or about 175 mg, or about 200 mg, or about 225 mg, or about 250 mg, or about 275 mg, or about 300 mg, or about 325 mg, or about 350 mg, or about 375 mg, or about 400 mg, or about 425 mg, or about 450 mg, or about 475 mg, or about 500 mg, or about 525 mg, or about 550 mg, or about 575 mg, or about 600 mg, or about 625 mg, or about 650 mg, or about 675 mg, or about 700 mg, or about 725 mg, or about 750 mg, or about 775 mg, or about 800 mg, or about 825 mg, or about 850 mg, or about 875 mg, or about 900 mg, or about 925 mg, or about 950 mg, or about 975 mg, or about 1000 mg, or about 1100 mg, or about 1200 mg, or about 1300 mg, or about 1400 mg, or about 1500 mg, or about 1600 mg, or about 1700 mg, or about 1800 mg, or about 1900 mg, or about 2000 mg.

In an embodiment is provided any of the methods described herein, wherein one or more cancer cells in said subject is determined to possess at least one molecular alteration in one or more of Tyro3, Axl, Mer, or c-Met prior to administration of said inhibitor of Tyro3, Axl, Mer, or c-Met.

In an embodiment is provided any of the methods described herein, wherein one or more cancer cells in said subject is determined to possess at least one molecular alteration in Tyro3 prior to administration of said inhibitor of Tyro3.

In an embodiment is provided any of the methods described herein, wherein one or more cancer cells in said subject is determined to possess at least one molecular alteration in Axl prior to administration of said inhibitor of Axl.

In an embodiment is provided any of the methods described herein, wherein one or more cancer cells in said subject is determined to possess at least one molecular alteration in Mer prior to administration of said inhibitor of Mer.

In an embodiment is provided any of the methods described herein, wherein one or more cancer cells in said subject is determined to possess at least one molecular alteration in c-Met prior to administration of said inhibitor of c-Met.

In an embodiment is provided any of the methods described herein, wherein said cancer is selected from heart sarcoma, lung cancer, small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal glands: neuroblastoma; and other tissues including connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

In an embodiment is provided any of the methods described herein, wherein said cancer is selected from lung cancer, small cell lung cancer (SCLC), non-small cell lung cancer (NSCLC), bronchogenic carcinoma, bronchial adenoma, lymphoma, chondromatous hamartoma, mesothelioma, stomach cancer gastric cancer, pancreatic cancer, cancer of the small bowel, Karposi's sarcoma, leiomyoma, hemangioma, lipoma, neurofibroma, fibroma, cancer of the large bowel, cancer of the genitourinary tract, kidney cancer, Wilm's tumor, nephroblastoma, leukemia, bladder cancer, urethral cancer, prostate cancer, ovarian cancer, cancer of the testis, liver cancer, breast cancer, hepatocellular carcinoma, cholangiocarcinoma, hepatoblastoma, angiosarcoma, hepatocellular adenoma, hemangioma, pancreatic endocrine tumors, pheochromocytoma, insulinoma, vasoactive intestinal peptide tumor, islet cell tumor, lucagonoma, bone cancer, osteogenic sarcoma (osteosarcoma), fibrosarcoma, malignant fibrous histiocytoma, chondrosarcoma, Ewing's sarcoma, malignant lymphoma, reticulum cell sarcoma, multiple myeloma, malignant giant cell tumor chordoma, osteochronfroma (osteocartilaginous exostoses), benign chondroma, chondroblastoma, chondromyxofibroma, osteoid osteoma, giant cell tumors, neoplasms of the central nervous system (CNS), primary CNS lymphoma, skull cancer, osteoma, hemangioma, granuloma, xanthoma, osteitis deformans, meninges, meningioma, meningiosarcoma, gliomatosis, brain cancer, astrocytoma, medulloblastoma, glioma, ependymoma, germinoma, pinealoma, glioblastoma multiform, oligodendroglioma, schwannoma, retinoblastoma, congenital tumors), spinal cord neurofibroma, meningioma, glioma, sarcoma), uterine cancer, endometrial carcinoma, cervical carcinoma, pre-tumor cervical dysplasia, ovarian carcinoma, serous cystadenocarcinoma, mucinous cystadenocarcinoma, unclassified carcinoma, granulosa-thecal cell tumors, Sertoli-Leydig cell tumors, dysgerminoma, malignant teratoma), cancer of the vulva (squamous cell carcinoma, intraepithelial carcinoma, adenocarcinoma, fibrosarcoma, melanoma), cancer of the vagina (clear cell carcinoma, squamous cell carcinoma, botryoid sarcoma (embryonal rhabdomyosarcoma), cancer of the fallopian tubes (carcinoma) myeloid leukemia, acute lymphoblastic leukemia, chronic lymphocytic leukemia, myeloproliferative diseases, multiple myeloma, myelodysplastic syndrome, Hodgkin's disease, non-Hodgkin's lymphoma, malignant lymphoma, cancer of the oral cavity, cancer of the parotid gland, cancer of the salivary glands, tonsil, oropharynx, nasopharynx, pyriform sinus, hypopharynx, skin cancer, malignant melanoma, cutaneous melanoma, basal cell carcinoma, squamous cell carcinoma, Karposi's sarcoma, dysplastic nevi, lipoma, angioma, dermatofibroma, cancer of the adrenal glands, neuroblastoma; ocular cancer, intraocular melanoma, and adnexa, breast cancer, cancer of the head and neck, anal cancer, thyroid cancer, parathyroid cancer, adrenal gland secondary and unspecified malignant neoplasm of lymph nodes, secondary malignant neoplasm of respiratory and digestive systems and secondary malignant neoplasm of other sites.

In an embodiment is provided any of the methods described herein, wherein said cancer is selected from lung cancer, small cell lung cancer (SCLC), non-small cell lung cancer (NSCLC), lymphoma, chondromatous hamartoma, mesothelioma, stomach cancer, gastric cancer, pancreatic cancer, Karposi's sarcoma, kidney cancer, Wilm's tumor, nephroblastoma, leukemia, bladder cancer, urethral cancer, prostate cancer, ovarian cancer, cancer of the testis, liver cancer, breast cancer, hepatocellular carcinoma, cholangiocarcinoma, hepatoblastoma, angiosarcoma, hepatocellular adenoma, hemangioma, fibrosarcoma, Ewing's sarcoma, malignant lymphoma, reticulum cell sarcoma, multiple myeloma, brain cancer, astrocytoma, medulloblastoma, glioma, ependymoma, germinoma, pinealoma, glioblastoma multiform, oligodendroglioma, schwannoma, retinoblastoma, glioma, uterine cancer, endometrial carcinoma, cervical carcinoma, pre-tumor cervical dysplasia, ovarian carcinoma, myeloid leukemia, acute lymphoblastic leukemia, chronic lymphocytic leukemia, myeloproliferative diseases, multiple myeloma, myelodysplastic syndrome, Hodgkin's disease, non-Hodgkin's lymphoma, malignant lymphoma, malignant melanoma, cutaneous melanoma, basal cell carcinoma, squamous cell carcinoma, Karposi's sarcoma, neuroblastoma, breast cancer, cancer of the head and neck, anal cancer, thyroid cancer, and parathyroid cancer.

In an embodiment is provided any of the methods described herein, wherein said cancer is selected from lung cancer (NSCLC and SCLC), cancer of the head or neck, ovarian cancer, colon cancer, rectal cancer, prostate cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non-Hodgkins' s lymphoma, and spinal axis tumors.

In an embodiment is provided a kit, comprising:
(a) a first composition comprising an inhibitor of Tyro3, Axl, Mer, or c-Met;
(b) a second composition comprising an anti-PD-1 agent or an anti-PD-L1 agent; and
(c) instructions for use of said first composition and said second composition in the treatment of cancer in a subject.

In an embodiment is provided a medicament for use in treating cancer in a subject, comprising a first composition comprising an inhibitor of Tyro3, Axl, Mer, or c-Met and a second composition comprising an anti-PD-1 agent or an anti-PD-L1 agent.

In an embodiment is provided a combination for use in treating a cancer in a subject, said combination comprising (a) an inhibitor of Tyro3, Axl, Mer, or c-Met, and (b) an anti-PD-1 agent or an anti-PD-L1 agent.

In an embodiment is provided a combination for use in treating a cancer in a subject, said combination comprising (a) a first pharmaceutical composition comprising an inhibitor of Tyro3, Axl, Mer, or c-Met, and (b) a second pharmaceutical composition comprising an anti-PD-1 agent or an anti-PD-L1 agent.

In an embodiment is provided any of the combinations for use described herein, wherein one or more cancer cells in said subject is determined to possess at least one molecular alteration in one or more of Tyro3, Axl, Mer, or c-Met prior to administration of said inhibitor of Tyro3, Axl, Mer, or c-Met.

In an embodiment is provided any of the combinations for use described herein, wherein one or more cancer cells in said subject is determined to possess at least one molecular alteration in one or more of Tyro3, Axl, Mer, or c-Met, comprising administering to said subject a therapeutically effective amount of a combination comprising (a) an inhibitor of Tyro3, Axl, Mer, or c-Met, and (b) an anti-PD-1 agent or an anti-PD-L1 agent.

In an embodiment is provided any of the combinations for use described herein, wherein said combination comprises an inhibitor of Tyro3 and an anti-PD-1 agent or an anti-PD-L1 agent.

In an embodiment is provided any of the combinations for use described herein, wherein said combination comprises an inhibitor of Axl and an anti-PD-1 agent or an anti-PD-L1 agent.

In an embodiment is provided any of the combinations for use described herein, wherein said combination comprises an inhibitor of Mer and an anti-PD-1 agent or an anti-PD-L1 agent.

In an embodiment is provided any of the combinations for use described herein, wherein said combination comprises an inhibitor of c-Met and an anti-PD-1 agent or an anti-PD-L1 agent.

In an embodiment is provided any of the combinations for use described herein, wherein said combination comprises N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, or a pharmaceutically acceptable salt thereof, and an anti-PD-1 agent or an anti-PD-L1 agent.

In an embodiment is provided any of the combinations for use described herein, wherein said anti-PD-1 agent or anti-PD-L1 agent is a monoclonal antibody.

In an embodiment is provided any of the combinations for use described herein, wherein said monoclonal antibody is a fully human monoclonal antibody.

In an embodiment is provided any of the combinations for use described herein, wherein said anti-PD-1 agent or anti-PD-L1 agent is selected from nivolumab, pembrolizumab, atezolizumab, avelumab, and durvalumab.

In an embodiment is provided any of the combinations for use described herein, wherein said anti-PD-1 agent or anti-PD-L1 agent is nivolumab.

In an embodiment is provided any of the combinations for use described herein, wherein anti-PD-1 agent or anti-PD-L1 agent is pembrolizumab.

In an embodiment is provided any of the combinations for use described herein, wherein said anti-PD-1 agent or anti-PD-L1 agent is atezolizumab.

In an embodiment is provided any of the combinations for use described herein, wherein said anti-PD-1 agent or anti-PD-L1 agent is avelumab.

In an embodiment is provided any of the combinations for use described herein, wherein said anti-PD-1 agent or anti-PD-L1 agent is durvalumab.

In an embodiment is provided any of the combinations for use described herein, wherein said inhibitor of Tyro3, Axl, Mer, or c-Met and said anti-PD-1 agent or anti-PD-L1 agent are administered to said subject simultaneously.

In an embodiment is provided any of the combinations for use described herein, wherein said inhibitor of Tyro3, Axl, Mer, or c-Met and said anti-PD-1 agent or anti-PD-L1 agent are administered to said subject sequentially.

In an embodiment is provided any of the combinations for use described herein, wherein said inhibitor of Tyro3, Axl, Mer, or c-Met is administered to said subject orally.

In an embodiment is provided any of the combinations for use described herein, wherein said anti-PD-1 agent or anti-PD-L1 agent is administered to said subject intravenously.

In an embodiment is provided any of the combinations for use described herein, wherein said anti-PD-1 agent or anti-PD-L1 agent is administered to said subject intraperitoneally.

In an embodiment is provided any of the combinations for use described herein, wherein said inhibitor of Tyro3, Axl, Mer, or c-Met is administered to said subject orally and said anti-PD-1 agent or anti-PD-L1 agent is administered to said subject intravenously.

In an embodiment is provided any of the combinations for use described herein, wherein said inhibitor of Tyro3, Axl, Mer, or c-Met is administered to said subject orally and said anti-PD-1 agent or anti-PD-L1 agent is administered to said subject intraperitoneally.

In an embodiment is provided any of the combinations for use described herein, wherein said anti-PD-1 agent or anti-PD-L1 agent is administered to said subject every 3 weeks.

In an embodiment is provided any of the combinations for use described herein, wherein said anti-PD-1 agent or anti-PD-L1 agent is administered to said subject in four doses every 3 weeks.

In an embodiment is provided any of the combinations for use described herein, wherein said anti-PD-1 agent or anti-PD-L1 agent is administered to said subject every week.

In an embodiment is provided any of the combinations for use described herein, wherein said anti-PD-1 agent or anti-PD-L1 agent is administered to said subject two doses every week.

In an embodiment is provided any of the combinations for use described herein, wherein said anti-PD-1 agent or anti-PD-L1 agent is administered to said subject in a dose of 3 mg per kilogram of the weight of said subject.

In an embodiment is provided any of the combinations for use described herein, wherein said anti-PD-1 agent or anti-PD-L1 agent is administered to said subject every 3 weeks.

In an embodiment is provided any of the combinations for use described herein, wherein said anti-PD-1 agent or anti-PD-L1 agent is administered to said subject in a dose of 3 mg per kilogram of the weight of said subject every 3 weeks.

In an embodiment is provided any of the combinations for use described herein, wherein said anti-PD-1 agent or anti-PD-L1 agent agent is administered to said subject in a dose of 3 mg per kilogram of the weight of said subject every 3 weeks for a total of 4 doses.

In an embodiment is provided any of the combinations for use described herein, wherein said anti-PD-1 agent or anti-PD-L1 agent is administered to said subject in a dose of 10 mg per kilogram of the weight of said subject.

In an embodiment is provided any of the combinations for use described herein, wherein said anti-PD-1 agent or anti-PD-L1 agent is administered to said subject twice every week.

In an embodiment is provided any of the combinations for use described herein, wherein said anti-PD-1 agent or anti-PD-L1 agent is administered to said subject in a dose of 10 mg per kilogram of the weight of said subject twice every week.

In an embodiment is provided any of the combinations for use described herein, wherein said anti-PD-1 agent or anti-PD-L1 agent agent is administered to said subject in a dose of 10 mg per kilogram of the weight of said subject every week for a total of 2 doses.

In an embodiment is provided any of the combinations for use described herein, wherein said inhibitor of Tyro3, Axl, Mer, or c-Met is administered to said subject at least once per day. In an embodiment is provided any of the combinations for use described herein, wherein said inhibitor of Tyro3, Axl, Mer, or c-Met is administered to said subject once per day, or twice per day, or three times per day, or four times per day, or five times per day, or six times per day, or seven times per day, or eight times per day, or nine times per day, or ten times per day.

In an embodiment is provided any of the combinations for use described herein, wherein said inhibitor of Tyro3, Axl, Mer, or c-Met is administered to said subject in a dose of about 0.1 mg per kilogram of subject weight to about 1000 mg per kilogram of subject weight. In an embodiment is provided any of the combinations for use described herein, wherein said inhibitor of Tyro3, Axl, Mer, or c-Met is administered to said subject in a dose of about 0.1 mg per kilogram of subject weight to about 1000 mg per kilogram of subject weight. In an embodiment is provided any of the methods described herein, wherein said inhibitor of Tyro3, Axl, Mer, or c-Met is administered to said subject in a dose of about 0.1 mg per kilogram of subject weight to about 750 mg per kilogram of subject weight, or from about 0.1 mg per kilogram of subject weight to about 650 mg per kilogram of subject weight, or from about 0.1 mg per kilogram of subject weight to about 575 mg per kilogram of subject weight, or from about 0.1 mg per kilogram of subject weight to about 550 mg per kilogram of subject weight, or from about 0.1 mg per kilogram of subject weight to about 525 mg per kilogram of subject weight, or from about 0.1 mg per kilogram of subject weight to about 500 mg per kilogram of subject weight, or from about 0.1 mg per kilogram of subject weight to about 475 mg per kilogram of subject weight, or from about 0.1 mg per kilogram of subject weight to about 450 mg per kilogram of subject weight, or from about 0.1 mg per kilogram of subject weight to about 425 mg per kilogram of subject weight, or from about 0.1 mg per kilogram of subject weight to about 400 mg per kilogram of subject weight, or from about 0.1 mg per kilogram of subject weight to about 375 mg per kilogram of subject weight, or from about 0.1 mg per kilogram of subject weight to about 350 mg per kilogram of subject weight, or from about 0.1 mg per kilogram of subject weight to about 325 mg per kilogram of subject weight, or from about 0.1 mg per kilogram of subject weight to about 300 mg per kilogram of subject weight, or from about 0.1 mg per kilogram of subject weight to about 275 mg per kilogram of subject weight, or from about 0.1 mg per kilogram of subject weight to about 250 mg per kilogram of subject weight, or from about 0.1 mg per kilogram of subject weight to about 225 mg per kilogram of subject weight, or from about 0.1 mg per kilogram of subject weight to about 200 mg per kilogram of subject weight, or from about 0.1 mg per kilogram of subject weight to about 175 mg per kilogram of subject weight, or from about 0.1 mg per kilogram of subject weight to about 150 mg per kilogram of subject weight, or from about 0.1 mg per kilogram of subject weight to about 125 mg per kilogram of subject weight, or from about 0.1 mg per kilogram of subject weight to about 100 mg per kilogram of subject weight, or from about 0.1 mg per kilogram of subject weight to about 75 mg per kilogram of subject weight, or from about 0.1 mg per kilogram of subject weight to about 50 mg per kilogram of subject weight, or from about 0.1 mg per kilogram of subject weight to about 25 mg per kilogram of subject weight, or from about 0.1 mg per kilogram of subject weight to about 20 mg per kilogram of subject weight, or from about 0.1 mg per kilogram of subject weight to about 15 mg per kilogram of subject weight, or from about 0.1 mg per kilogram of subject weight to about 10 mg per kilogram of subject weight, or from about 0.1 mg per kilogram of subject weight to about 5 mg per kilogram of subject weight, or from about 0.1 mg per kilogram of subject weight to about 2.5 mg per kilogram of subject weight, or from about 0.1 mg per kilogram of subject weight to about 2 mg per kilogram of subject weight, or from about 0.1 mg per kilogram of subject weight to about 1 mg per kilogram of subject weight.

In an embodiment is provided any of the combinations for use described herein, wherein said inhibitor of Tyro3, Axl, Mer, or c-Met is administered to said subject in a dose of from about 0.1 mg per kilogram of subject weight to about 30 mg per kilogram of subject weight.

In an embodiment is provided any of the combinations for use described herein, wherein said inhibitor of Tyro3, Axl, Mer, or c-Met is administered to said subject in a dose of about 1 mg, or about 5 mg, or about 10 mg, or about 15 mg, or about 20 mg, or about 25 mg, or about 30 mg, or about 35 mg, or about 40 mg, or about 45 mg, or about 50 mg, or about 55 mg, or about 60 mg, or about 65 mg, or about 70 mg, or about 75 mg, or about 80 mg, or about 85 mg, or about 90 mg, or about 95 mg, or about 100 mg, or about 125 mg, or about 150 mg, or about 175 mg, or about 200 mg, or about 225 mg, or about 250 mg, or about 275 mg, or about 300 mg, or about 325 mg, or about 350 mg, or about 375 mg, or about 400 mg, or about 425 mg, or about 450 mg, or about 475 mg, or about 500 mg, or about 525 mg, or about 550 mg, or about 575 mg, or about 600 mg, or about 625 mg, or about 650 mg, or about 675 mg, or about 700 mg, or about 725 mg, or about 750 mg, or about 775 mg, or about 800 mg, or about 825 mg, or about 850 mg, or about 875 mg, or about 900 mg, or about 925 mg, or about 950 mg, or about 975 mg, or about 1000 mg, or about 1100 mg, or about 1200 mg, or about 1300 mg, or about 1400 mg, or about 1500 mg, or about 1600 mg, or about 1700 mg, or about 1800 mg, or about 1900 mg, or about 2000 mg.

In an embodiment is provided any of the combinations for use described herein, wherein said inhibitor of Tyro3, Axl, Mer, or c-Met is N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, or a pharmaceutically acceptable salt thereof.

In an embodiment is provided any of the combinations for use described herein, wherein one or more cancer cells in said subject is determined to possess at least one molecular alteration in one or more of Tyro3, Axl, Mer, or c-Met prior to administration of said inhibitor of Tyro3, Axl, Mer, or c-Met.

In an embodiment is provided any of the combinations for use described herein, wherein one or more cancer cells in said subject is determined to possess at least one molecular alteration in Tyro3 prior to administration of said inhibitor of Tyro3.

In an embodiment is provided any of the combinations for use described herein, wherein one or more cancer cells in said subject is determined to possess at least one molecular alteration in Axl prior to administration of said inhibitor of Axl.

In an embodiment is provided any of the combinations for use described herein, wherein one or more cancer cells in said subject is determined to possess at least one molecular alteration in Mer prior to administration of said inhibitor of Mer.

In an embodiment is provided any of the combinations for use described herein, wherein one or more cancer cells in said subject is determined to possess at least one molecular alteration in c-Met prior to administration of said inhibitor of c-Met.

In an embodiment is provided any of the combinations for use described herein, wherein said cancer is selected from heart sarcoma, lung cancer, small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal glands: neuroblastoma; and other tissues including connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

In an embodiment is provided any of the combinations for use described herein, wherein said cancer is selected from lung cancer, small cell lung cancer (SCLC), non-small cell lung cancer (NSCLC), bronchogenic carcinoma, bronchial adenoma, lymphoma, chondromatous hamartoma, mesothelioma, stomach cancer gastric cancer, pancreatic cancer, cancer of the small bowel, Karposi's sarcoma, leiomyoma, hemangioma, lipoma, neurofibroma, fibroma, cancer of the large bowel, cancer of the genitourinary tract, kidney cancer, Wilm's tumor, nephroblastoma, leukemia, bladder cancer, urethral cancer, prostate cancer, ovarian cancer, cancer of the testis, liver cancer, breast cancer, hepatocellular carcinoma, cholangiocarcinoma, hepatoblastoma, angiosarcoma, hepatocellular adenoma, hemangioma, pancreatic endocrine tumors, pheochromocytoma, insulinoma, vasoactive intestinal peptide tumor, islet cell tumor, lucagonoma, bone cancer, osteogenic sarcoma (osteosarcoma), fibrosarcoma, malignant fibrous histiocytoma, chondrosarcoma, Ewing's sarcoma, malignant lymphoma, reticulum cell sarcoma, multiple myeloma, malignant giant cell tumor chordoma, osteochronfroma (osteocartilaginous exostoses), benign chondroma, chondroblastoma, chondromyxofibroma, osteoid osteoma, giant cell tumors, neoplasms of the central nervous system (CNS), primary CNS lymphoma, skull cancer, osteoma, hemangioma, granuloma, xanthoma, osteitis deformans, meninges, meningioma, meningiosarcoma, gliomatosis, brain cancer, astrocytoma, medulloblastoma, glioma, ependymoma, germinoma, pinealoma, glioblastoma multiform, oligodendroglioma, schwannoma, retinoblastoma, congenital tumors), spinal cord neurofibroma, meningioma, glioma, sarcoma), uterine cancer, endometrial carcinoma, cervical carcinoma, pre-tumor cervical dysplasia, ovarian carcinoma, serous cystadenocarcinoma, mucinous cystadenocarcinoma, unclassified carcinoma, granulosa-thecal cell tumors, Sertoli-Leydig cell tumors, dysgerminoma, malignant teratoma), cancer of the vulva (squamous cell carcinoma, intraepithelial carcinoma, adenocarcinoma, fibrosarcoma, melanoma), cancer of the vagina (clear cell carcinoma, squamous cell carcinoma, botryoid sarcoma (embryonal rhabdomyosarcoma), cancer of the fallopian tubes (carcinoma) myeloid leukemia, acute lymphoblastic leukemia, chronic lymphocytic leukemia, myeloproliferative diseases, multiple myeloma, myelodysplastic syndrome, Hodgkin's disease, non-Hodgkin's lymphoma, malignant lymphoma, cancer of the oral cavity, cancer of the parotid gland, cancer of the salivary glands, tonsil, oropharynx, nasopharynx, pyriform sinus, hypopharynx, skin cancer, malignant melanoma, cutaneous melanoma, basal cell carcinoma, squamous cell carcinoma, Karposi's sarcoma, dysplastic nevi, lipoma, angioma, dermatofibroma, cancer of the adrenal glands, neuroblastoma; ocular cancer, intraocular melanoma, and adnexa, breast cancer, cancer of the head and neck, anal cancer, thyroid cancer, parathyroid cancer, adrenal gland secondary and unspecified malignant neoplasm of lymph nodes, secondary malignant neoplasm of respiratory and digestive systems and secondary malignant neoplasm of other sites.

In an embodiment is provided any of the combinations for use described herein, wherein said cancer is selected from lung cancer, small cell lung cancer (SCLC), non-small cell lung cancer (NSCLC), lymphoma, chondromatous hamartoma, mesothelioma, stomach cancer, gastric cancer, pancreatic cancer, Karposi's sarcoma, kidney cancer, Wilm's tumor, nephroblastoma, leukemia, bladder cancer, urethral cancer, prostate cancer, ovarian cancer, cancer of the testis, liver cancer, breast cancer, hepatocellular carcinoma, cholangiocarcinoma, hepatoblastoma, angiosarcoma, hepatocellular adenoma, hemangioma, fibrosarcoma, Ewing's sarcoma, malignant lymphoma, reticulum cell sarcoma, multiple myeloma, brain cancer, astrocytoma, medulloblastoma, glioma, ependymoma, germinoma, pinealoma, glioblastoma multiform, oligodendroglioma, schwannoma, retinoblastoma, glioma, uterine cancer, endometrial carcinoma, cervical carcinoma, pre-tumor cervical dysplasia, ovarian carcinoma, myeloid leukemia, acute lymphoblastic leukemia, chronic lymphocytic leukemia, myeloproliferative diseases, multiple myeloma, myelodysplastic syndrome, Hodgkin's disease, non-Hodgkin's lymphoma, malignant lymphoma, malignant melanoma, cutaneous melanoma, basal cell carcinoma, squamous cell carcinoma, Karposi's sarcoma, neuroblastoma, breast cancer, cancer of the head and neck, anal cancer, thyroid cancer, and parathyroid cancer.

In an embodiment is provided any of the combinations for use described herein, wherein said cancer is selected from lung cancer (NSCLC and SCLC), cancer of the head or neck, ovarian cancer, colon cancer, rectal cancer, prostate cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non-Hodgkins' s lymphoma, and spinal axis tumors.

In another embodiment are provided therapeutic methods and uses comprising administering one or more compounds disclosed herein, or a pharmaceutically acceptable salt thereof, or a pharmaceutically acceptable salt thereof, alone or in combination with another therapeutic or palliative agent to a mammal in need of such treatment. In an embodiment, the mammal is a human. In other embodiments, the mammal is a dog or cat.

In another embodiment are provided methods for the treatment of abnormal cell growth in a mammal comprising administering to a mammal a therapeutically effective amount of one or more compounds disclosed herein, or a pharmaceutically acceptable salt thereof, or a pharmaceutically acceptable salt thereof.

In an embodiment is provided a method of treating cancer in a subject, comprising administering to said subject a therapeutically effective amount of a combination comprising (a) an inhibitor of Tyro3, Axl, Mer, or c-Met, (b) an anti-PD-1 agent or an anti-PD-L1 agent, and (c) an anti-tumor agent, which amounts are together effective in treating said abnormal cell growth. In some embodiments, the anti-tumor agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

In an embodiment is provided a method of treating cancer in a subject, comprising administering to said subject a therapeutically effective amount of a combination comprising (a) an inhibitor of Tyro3, Axl, Mer, or c-Met, (b) an anti-PD-1 agent or an anti-PD-L1 agent, and (c) an anti-cancer therapeutic agent or a palliative agent, which amounts are together effective in treating said cancer. In some such embodiments, one or more anti-cancer therapeutic agent are selected from anti-tumor agents, anti-angiogenesis agents, signal transduction inhibitors and antiproliferative agents, which amounts are together effective in treating said cancer.

In an embodiment is provided a method of treating cancer in a subject, comprising administering to said subject a therapeutically effective amount of a combination comprising (a) an inhibitor of Tyro3, Axl, Mer, or c-Met, (b) an anti-PD-1 agent or an anti-PD-L1 agent, and (c) one or more substances selected from anti-tumor agents, anti-angiogenesis agents, signal transduction inhibitors and antiproliferative agents, which amounts are together effective in treating said cancer.

Each of the embodiments of the compounds disclosed herein can be combined with one or more other embodiments of the compounds described herein that is not inconsistent with the embodiment(s) with which it is combined. In addition, each of the embodiments disclosed herein envisions within its scope the pharmaceutically acceptable salts of the compounds disclosed herein. Accordingly, the phrase “or a pharmaceutically acceptable salt thereof” is implicit in the description of all compounds described herein.

In another embodiment are provided methods to treat cancer and cell proliferative disorders.

In another embodiment are provided methods to treat specific types of cancer including carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, angiosarcoma, glioblastoma, holangiocarcinoma, inflammatory myofibroblastic tumor, epitheloid hemangioendothelioma, astrocytoma, meningioma, angiosarcoma, epitheloid hemangiothelioma, keratocanthomas, thyroid follicular cancer, Kaposi's sarcoma, and Pancreatic cancer.

In another embodiment are provided methods to treat specific types of cancer such as, but not restricted to, breast cancer, lung cancer, colorectal cancer, prostate cancer, ovarian cancer, endometrial cancer, gastric cancer, clear cell renal cell carcinoma, invasive ductal carcinoma (breast), uveal melanoma, multiple myeloma, rhabdomyosarcoma, Ewing's sarcoma, Kaposi's sarcoma, Pancreatic cancer, and medulloblastoma.

In another embodiment are provided methods to treat cell proliferative disorders such as, but not restricted to, benign prostate hyperplasia, familial adenomatosis polyposis, neuro-fibromatosis, psoriasis, atherosclerosis and conditions involving vascular smooth muscle proliferation or neointimal formation such as restenosis following angioplasty or surgery, pulmonary fibrosis, arthritis, glomerulonephritis, retinopathies including diabetic and neonatal retinopathies and age related macular degeneration, graft vessel disease, such as can occur following vessel or organ transplantation, acromegaly and disorders secondary to acromegaly as well as other hypertrophic conditions in which IGF/IGF-1R signaling is implicated, such as fibrotic lung disease, pathologies related to chronic or acute oxidative stress or hyperoxia induced tissue damage, and metabolic disorders in which elevated IGF levels or IGF-1R activity are implicated, such as obesity.

In another embodiment are provided methods of affecting tumor angiogenesis and metastasis inhibition.

In some embodiments, the one or more molecular alterations detected in the biological sample involve at least two, at least three, or at least four of the biomarkers. In some embodiments, the knowledge of the presence of the one or more molecular alterations in the biological sample is acquired from an assay that includes contacting the biological sample with one or more antibodies or fragments thereof specific for the biomarkers. In some embodiments, the specific antibodies are monoclonal antibodies. In some embodiments, the biological sample is contacted with one or more of the specific antibodies simultaneously. In some embodiments, the biological sample is sequentially contacted with the specific antibodies. In some embodiments, the one or more molecular alterations results in elevated expression of one or more of the Tyro3, Axl, Mer, or c-Met biomarkers. In some embodiments, the knowledge of the one or more molecular alterations is acquired from an assay wherein determining whether the expression of one or more biomarker is elevated includes: (a) determining the expression level of the one or more biomarkers in the biological sample; and (b) comparing the determined expression level to a reference expression level. In some embodiments, the knowledge of the one or more molecular alterations is acquired from an antibody-based assay. In some embodiments, the antibody-based assay is selected from the group consisting of ELISA, immunohistochemistry, western blotting, mass spectrometry, flow cytometry, protein-microarray, immunofluorescence, and a multiplex detection assay. In some embodiments, the antibody-based assay includes an immunohistochemistry analysis.

In some embodiments, implementations of the methods disclosed herein include acquiring knowledge of a genetic alteration in the cancer of the subject from a second analytical assay prior to the administering step, wherein the second analytical assay is selected from the group consisting of capillary electrophoresis, nucleic acid sequencing, polypeptide sequencing, restriction digestion, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), and a kinase activity assay. In some embodiments, the cancer is cancer is selected from the group consisting of anaplastic large-cell lymphoma (ALCL), colorectal cancer (CRC), cholangiocarcinoma, gastric, glioblastomas (GBM), leiomyosarcoma, melanoma, non-small cell lung cancer (NSCLC), squamous cell lung cancer, neuroblastoma (NB), ovarian cancer, pancreatic cancer, prostate cancer, medullary thyroid cancer, breast cancer, and papillary thyroid cancer. In some embodiments, the knowledge of the one or more molecular alterations is obtained from an assay performed simultaneously on a plurality of biological samples. In some embodiments, the plurality of biological samples includes at least 6, 12, 24, 48, 96, 200, 384, 400, 500, 1000, 1500, or 3000 samples. In some embodiments, the one or more molecular alterations is selected from a genetic mutation, a gene amplification, a gene rearrangement, a single-nucleotide variation (SNV), a deletion, an insertion, an InDel mutation, a single nucleotide point mutation (SNP), an epigenetic alteration, a splicing variant, an RNA/protein overexpression, an aberrant RNA/protein expression, and any combination thereof. In some embodiments, the one or more molecular alterations include an insertion of a heterologous nucleic acid sequence within a coding sequence of a biomarker gene. In some embodiments, the insertion forms a chimeric nucleic acid sequence that encodes a fusion peptide. In some embodiments, the acquiring knowledge of the one or more molecular alterations further includes determining a nucleic acid sequence and/or an amino acid sequence comprising the one or more molecular alterations.

Some embodiments provide a pharmaceutical composition comprising one or more compounds disclosed herein, or a pharmaceutically acceptable salt thereof, in combination with one or more chemotherapeutic agents or radiotherapy, such as radiotherapy as commonly administered to treat, ameliorate the symptoms of, or prevent or delay the onset of cancer. Such agents can include, but are not limited to, antihormonal agents such as antiestrogens, antiandrogens and aromatase inhibitors, topoisomerase I inhibitors, topoisomerase II inhibitors, agents that target microtubules, platin-based agents, alkylating agents, DNA damaging or intercalating agents, antineoplastic antimetabolites, other kinase inhibitors, other anti-angiogenic agents, inhibitors of kinesins, therapeutic monoclonal antibodies, inhibitors of mTOR, histone deacetylase inhibitors, farnesyl transferase inhibitors, and inhibitors of hypoxic response.

Some embodiments provide a product or kit comprising one or more compounds disclosed herein, or a pharmaceutically acceptable salt thereof, and one or more chemotherapeutic agents, as a combined preparation for simultaneous, separate or sequential use in anticancer therapy.

Some embodiments provide one or more compounds as disclosed herein, or a pharmaceutically acceptable salt thereof, for use as a medicament.

Some embodiments provide the use of one or more compounds as disclosed herein, or a pharmaceutically acceptable salt thereof, in the manufacture of a medicament with antitumor activity.

Some embodiments include any of the methods described herein, wherein said cancer is selected from non-small cell lung cancer, papillary thyroid cancer, neuroblastoma, pancreatic cancer and colorectal cancer. Some embodiments are any of the methods described herein wherein said cancer is non-small cell lung cancer. Some embodiments include any of the methods described herein, wherein said cancer is said cancer is papillary thyroid cancer. Some embodiments include any of the methods described herein, wherein said cancer is wherein said cancer is neuroblastoma. Some embodiments include any of the methods described herein, wherein said cancer is wherein said cancer is pancreatic cancer. Some embodiments include any of the methods described herein, wherein said cancer is wherein said cancer is colorectal cancer.

Unless indicated otherwise, all references herein to compounds disclosed herein, or a pharmaceutically acceptable salt thereof, include references to salts, solvates, hydrates and complexes thereof, and to solvates, hydrates and complexes of salts thereof, including polymorphs, stereoisomers, and isotopically labeled versions thereof.

The compounds disclosed herein may exist in the form of pharmaceutically acceptable salts such as, e.g., acid addition salts and base addition salts of the compounds of one of the formulae provided herein. As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the compounds of the formulae disclosed herein.

For example, the compounds disclosed herein that are basic in nature are capable of forming a wide variety of salts with various inorganic and organic acids. Although such salts must be pharmaceutically acceptable for administration to mammals, it is often desirable in practice to initially isolate the compounds disclosed herein from the reaction mixture as a pharmaceutically unacceptable salt and convert the latter to a free base and subsequently to a pharmaceutically acceptable acid addition salt. The acid addition salts of the base compounds disclosed herein can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

The acids that may be used to prepare pharmaceutically acceptable acid addition salts of such basic compounds of those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate and pamoate [i.e., 1,1′-methylene-bis-(2-hydroxy-3-naphthoate)] salts.

Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, .gamma.-hydroxybutyrate, iodide, isobutyrate, isothionate, lactate, lactobionate, laurate, malate, maleate, malonate, mandelate, mesylate, metaphosphate, methane-sulfonate, methyl sulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phosphate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The compounds disclosed herein that include a basic moiety, such as an amino group, may form pharmaceutically acceptable salts with various amino acids, in addition to the acids mentioned above.

Those compounds disclosed herein that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of the compounds disclosed herein are those which form non-toxic base salts with the acidic compounds herein. These salts may be prepared by any suitable method, for example, treatment of the free acid with an inorganic or organic base, such as an amine (primary, secondary or tertiary), an alkali metal hydroxide or alkaline earth metal hydroxide, or the like. These salts can also be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The chemical bases that may be used as reagents to prepare pharmaceutically acceptable base salts of the compounds disclosed herein that are acidic in nature are those that form non-toxic base salts with such compounds. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate and hemicalcium salts.

For a review on suitable salts, see “Handbook of Pharmaceutical Salts: Properties, Selection, and Use” by Stahl and Wermuth (Wiley-VCH, Weinheim, Germany, 2002).

Salts of the compounds disclosed herein can be prepared according to methods known to those of skill in the art. A pharmaceutically acceptable salt of the inventive compounds can be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized.

It will be understood by those of skill in the art that the compounds disclosed herein in free base form having a basic functionality may be converted to the acid addition salts by treating with a stoichiometric excess of the appropriate acid. The acid addition salts of the compounds disclosed herein may be reconverted to the corresponding free base by treating with a stoichiometric excess of a suitable base, such as potassium carbonate or sodium hydroxide, typically in the presence of aqueous solvent, and at a temperature of between about 0° C. and 100° C. The free base form may be isolated by conventional means, such as extraction with an organic solvent. In addition, acid addition salts of the compounds disclosed herein may be interchanged by taking advantage of differential solubilities of the salts, volatilities or acidities of the acids, or by treating with the appropriately loaded ion exchange resin. For example, the interchange may be affected by the reaction of a salt of the compounds disclosed herein with a slight stoichiometric excess of an acid of a lower pK than the acid component of the starting salt. This conversion is typically carried out at a temperature between about 0° C. and the boiling point of the solvent being used as the medium for the procedure. Similar exchanges are possible with base addition salts, typically via the intermediacy of the free base form.

Pharmaceutically acceptable salts of the compounds disclosed herein may be prepared by one or more of the following methods: (i) by reacting the compound disclosed herein with the desired acid or base; (ii) by removing an acid- or base-labile protecting group from a suitable precursor of the compound disclosed herein or by ring-opening a suitable cyclic precursor, for example, a lactone or lactam, using the desired acid or base; or (iii) by converting one salt of the compound disclosed herein to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds disclosed herein may exist in both unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm. The term ‘solvate’ is used herein to describe a molecular complex comprising the compound disclosed herein and one or more pharmaceutically acceptable solvent molecules, for example, ethanol. The term ‘hydrate’ is employed when the solvent is water. Pharmaceutically acceptable solvates in accordance with the embodiments disclosed herein include hydrates and solvates wherein the solvent of crystallization may be isotopically substituted, e.g., D₂O, d₆-acetone, d₆-DMSO.

Also included within the scope disclosed herein are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see Haleblian, J. Pharm. Sci., 1975, 64 (8):1269-1288, the disclosure of which is incorporated herein by reference in its entirety.

Hereinafter all references to compounds disclosed herein include references to salts, solvates and complexes thereof and to solvates and complexes of salts thereof.

The compounds disclosed herein include all polymorphs and crystal habits thereof, prodrugs and isomers thereof (including optical, geometric and tautomeric isomers) as hereinafter defined and isotopically-labeled compounds disclosed herein.

The compounds disclosed herein may have asymmetric carbon atoms. The carbon-carbon bonds of the compounds disclosed herein may be depicted herein using a solid line, a solid wedge, or a dotted wedge. The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds disclosed herein may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds disclosed herein can exist as enantiomers and diastereomers or as racemates and mixtures thereof. The use of a solid line to depict bonds to one or more asymmetric carbon atoms in one or more compounds disclosed herein and the use of a solid or dotted wedge to depict bonds to other asymmetric carbon atoms in the same compound is meant to indicate that a mixture of diastereomers is present.

Compounds disclosed herein containing one or more asymmetric carbon atoms can exist as two or more stereoisomers, such as racemates, enantiomers, or diastereomers. Stereoisomers of the compounds of the formulae herein can include cis and trans isomers, optical isomers such as (R) and (S) enantiomers, diastereomers, geometric isomers, rotational isomers, atropisomers, conformational isomers, and tautomers of the compounds disclosed herein, including compounds exhibiting more than one type of isomerism; and mixtures thereof (such as racemates and diastereomeric pairs). Also included are acid addition or base addition salts wherein the counterion is optically active, for example, d-lactate or l-lysine, or racemic, for example, dl-tartrate or dl-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds disclosed herein may exhibit the phenomena of tautomerism and structural isomerism. For example, the compounds may exist in several tautomeric forms, including the enol and imine form, and the keto and enamine form and geometric isomers and mixtures thereof. All such tautomeric forms are included within the scope of compounds disclosed herein. Tautomers exist as mixtures of a tautomeric set in solution. In solid form, usually one tautomer predominates. Even though one tautomer may be described, the compounds disclosed herein are meant to encompass all tautomers of the compounds of the formulae provided.

In addition, some of the compounds disclosed herein may form atropisomers (e.g., substituted biaryls). Atropisomers are conformational stereoisomers which occur when rotation about a single bond in the molecule is prevented, or greatly slowed, as a result of steric interactions with other parts of the molecule and the substituents at both ends of the single bond are unsymmetrical. The interconversion of atropisomers is slow enough to allow separation and isolation under predetermined conditions. The energy barrier to thermal racemization may be determined by the steric hindrance to free rotation of one or more bonds forming a chiral axis.

Where one or more compounds disclosed herein contains an alkenyl or alkenylene group, geometric cis/trans (or Z/E) isomers are possible. Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art.

Chiral compounds disclosed herein (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art; see, for example, “Stereochemistry of Organic Compounds” by E L Eliel (Wiley, New York, 1994), the disclosure of which is incorporated herein by reference in its entirety.

As used herein, the term “enantiomerically pure” describes one or more compounds that is present as a single enantiomer and which is described in terms of enantiomeric excess (e.e.). Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, “diastereomerically pure” as used herein, describes one or more compounds that is present as a diastereomer and which is described in terms of diasteriomeric excess (d.e.). Preferably, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

In another embodiment are included isotopically-labeled compounds, which are identical to those recited in one of the formulae provided, but for the fact that one or more atoms are replaced by an atom having an atomic mass or mass number different from the atomic mass or mass number usually found in nature.

Isotopically-labeled compounds disclosed herein can generally be prepared by conventional techniques known to those skilled in the art or by processes analogous to those described herein, using an appropriate isotopically-labeled reagent in place of the non-labeled reagent otherwise employed.

Examples of isotopes that may be incorporated into compounds disclosed herein include isotopes of hydrogen, carbon, nitrogen, oxygen, phosphorus, fluorine and chlorine, such as, but not limited to, ²H, ³H, ¹³C, ¹⁴C, ¹⁵, ¹⁸O, ¹⁷O, ³¹P, ³²P, ³⁵P, ¹⁸F, and ³⁶Cl. Certain isotopically-labeled compounds disclosed herein, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds disclosed herein may generally be prepared by carrying out procedures to those of ordinary skill in the art. Pharmaceutically acceptable solvates in accordance with the present disclosure include those wherein the solvent of crystallization may be isotopically substituted, e.g., D₂O, d₆-acetone, d₆-DMSO.

Compounds disclosed herein intended for pharmaceutical use may be administered as crystalline or amorphous products, or mixtures thereof. They may be obtained, for example, as solid plugs, powders, or films by methods such as precipitation, crystallization, freeze drying, spray drying, or evaporative drying. Microwave or radio frequency drying may be used for this purpose.

Some embodiments relate to the use of any of the compounds as described herein, or a pharmaceutically acceptable salt thereof, in the manufacture of a medicament for the treatment of abnormal cell growth in a mammal. In another embodiment are provided the use of any of the compounds as described herein, or a pharmaceutically acceptable salt thereof, in the manufacture of a medicament for the treatment of abnormal cell growth in a mammal wherein the abnormal cell growth is cancerous or non-cancerous. In some embodiments, the abnormal cell growth is cancerous. In another embodiment, the abnormal cell growth is non-cancerous.

Some embodiments relate to any of the compounds described herein, or pharmaceutically acceptable salts thereof, for use as a medicament. Some embodiments relate to the use of any of the compounds described above, or pharmaceutically acceptable salts thereof, for the manufacture of a medicament for the treatment of abnormal cell growth.

Some embodiments relate to compositions comprising one or more compounds disclosed herein, or a pharmaceutically acceptable salt thereof (e.g., pharmaceutical compositions). In another embodiment are provided pharmaceutical compositions comprising one or more compounds disclosed herein, or a pharmaceutically acceptable salt, one or more pharmaceutically acceptable carriers and, optionally, at least one additional medicinal or pharmaceutical agent. In some embodiments, the at least one additional medicinal or pharmaceutical agent is an anti-cancer agent as described below.

The pharmaceutically acceptable carrier may comprise a conventional pharmaceutical carrier or excipient. Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages.

In some embodiments, the composition comprises a therapeutically effective amount of one or more compounds disclosed herein, or a pharmaceutically acceptable salt thereof, and one or more pharmaceutically acceptable carriers.

The compounds disclosed herein, or their pharmaceutically acceptable salts, may be formulated into pharmaceutical compositions as described below in any pharmaceutical form recognizable to the skilled artisan as being suitable. Pharmaceutical compositions disclosed herein comprise a therapeutically effective amount of at least one compound disclosed herein and an inert, pharmaceutically acceptable carrier or diluent.

To treat or prevent diseases or conditions mediated by Tyro3, Axl, Mer, or c-Met, or a combination thereof, a pharmaceutical composition disclosed herein is administered in a suitable formulation prepared by combining a therapeutically effective amount of at least one compound disclosed herein, or a pharmaceutically acceptable salt thereof, with one or more pharmaceutically suitable carriers, which may be selected, for example, from diluents, excipients and auxiliaries that facilitate processing of the active compounds into the final pharmaceutical preparations.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g., parenteral or oral administration.

To obtain a stable water-soluble dose form, one or more compounds disclosed herein, or a pharmaceutically acceptable salt thereof, may be dissolved in an aqueous solution of an organic or inorganic acid, such as a 0.3 M solution of succinic acid or citric acid. If a soluble salt form is not available, the compound or salt may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, one or more compounds disclosed herein, or a pharmaceutically acceptable salt thereof, is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds disclosed herein, or a pharmaceutically acceptable salt thereof, may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds disclosed herein, or a pharmaceutically acceptable salt thereof, can be formulated by combining the compound with pharmaceutically acceptable carriers known in the art. Such carriers enable the compounds disclosed herein to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present disclosure may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds disclosed herein, or a pharmaceutically acceptable salt thereof, may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the compounds disclosed herein, or a pharmaceutically acceptable salt thereof, may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the compounds disclosed herein, or a pharmaceutically acceptable salt thereof, may be in powder form for constitution with a suitable vehicle, e.g., sterile pyrogen-free water, before use.

In addition to the formulations described above, the compounds disclosed herein, or a pharmaceutically acceptable salt thereof, may also be formulated as a depot preparation. Such long-acting formulations may be administered by implantation (for example, subcutaneously or intramuscularly) or by intramuscular injection. Thus, for example, the compounds disclosed herein, or a pharmaceutically acceptable salt thereof, may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5 W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions disclosed herein may also comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire®, Capryol®, Labrafil®, Labrasol®, Lauroglycol®, Plurol®, Peceol®, Transcutol® and the like may be used.

Further, the pharmaceutical compositions disclosed herein may be incorporated into a skin patch for delivery of the drug directly onto the skin.

It will be appreciated that the actual dosages of the compounds disclosed herein, or pharmaceutically acceptable salts thereof, will vary according to the particular agent being used, the particular composition formulated, the mode of administration, and the particular site, host, and disease being treated. Those skilled in the art using conventional dosage-determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

This amount will vary depending upon a variety of factors, including but not limited to the characteristics of the bioactive compositions and formulations disclosed herein (including activity, pharmacokinetics, pharmacodynamics, and bioavailability thereof), the physiological condition of the subject treated (including age, sex, disease type and stage, general physical condition, responsiveness to a given dosage, and type of medication) or cells, the nature of the pharmaceutically acceptable carrier mg/kg or carriers in the formulation, and the route of administration. Further, an effective or therapeutically effective amount may vary depending on whether the one or more bioactive compositions and formulations disclosed herein is administered alone or in combination with other drug(s), other therapy/therapies or other therapeutic method(s) or modality/modalities. One skilled in the clinical and pharmacological arts will be able to determine an effective amount or therapeutically effective amount through routine experimentation, namely by monitoring a cell's or subject's response to administration of the one or more bioactive compositions and formulations disclosed herein and adjusting the dosage accordingly.

In some embodiments, a dose of one or more of the compounds disclosed herein, or a pharmaceutically acceptable salt thereof, may range from about 0.1 mg/kg to about 100 mg/kg or more, depending on the factors mentioned above. In other alternatives, the dosage may range from about 0.1 mg/kg to about 100 mg/kg; or about 1 mg/kg to about 100 mg/kg; or about 5 mg/kg up to about 100 mg/kg. For topical applications such as, for example, treatment of various hair conditions, according to some alternatives disclosed herein, suitable dosage may range from about 1 mg/kg to about 10 g/kg; or about 10 mg/kg to about 1 g/kg; or about 50 mg/kg up to about 10 g/kg. Additional guidance with regard to this aspect can be found in, for example, Remington: The Science and Practice of Pharmacy, 21st Edition, Univ. of Sciences in Philadelphia (USIP), Lippincott Williams & Wilkins, Philadelphia, Pa., 2005.

In another embodiment are provided pharmaceutically acceptable formulations comprising one or more compounds disclosed herein, or a salt or solvate thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg. Furthermore, the pharmaceutically acceptable formulations disclosed herein may contain one or more compounds disclosed herein, or a salt or solvate thereof, in an amount of about 50 mg, about 100 mg, about 150 mg, about 200 mg, about 250 mg, about 300 mg, about 350 mg, about 400 mg, about 450 mg, or about 500 mg.

In another embodiment are provided pharmaceutically acceptable formulations comprising one or more compounds disclosed herein, or a pharmaceutically acceptable salt thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds disclosed herein, or a pharmaceutically acceptable salt thereof, may be administered to a mammal suffering from abnormal cell growth, such as a human, either alone or as part of a pharmaceutically acceptable formulation, once a day, twice a day, three times a day, or four times a day, or even more frequently.

Those of ordinary skill in the art will understand that with respect to the compounds disclosed herein, or a pharmaceutically acceptable salt thereof, the particular pharmaceutical formulation, the dosage, and the number of doses given per day to a mammal requiring such treatment, are all choices within the knowledge of one of ordinary skill in the art and can be determined without undue experimentation.

Administration of the compounds disclosed herein, or a pharmaceutically acceptable salt thereof, may be effected by any method that enables delivery of the compounds to the site of action. These methods include oral routes, intraduodenal routes, parenteral injection (including intravenous, subcutaneous, intramuscular, intravascular or infusion), topical, and rectal administration.

Administration of the compounds disclosed herein, or a pharmaceutically acceptable salt thereof, may be effected by any method that enables delivery of the compounds to the site of action, including intraperitoneal routes.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of compounds disclosed herein, or a pharmaceutically acceptable salt thereof, calculated to produce the desired therapeutic effect in association with the required pharmaceutical carrier. The specification for the dosage unit forms disclosed herein are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the presently disclosed methods.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. The embodiments disclosed herein are intended to encompass intra-subject dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The compounds, compositions, combination and methods provided herein are useful for the treatment of cancers including but not limited to cancers of the: circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal glands: neuroblastoma; and other tissues including connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

More specifically, examples of cancer when used herein in connection with compounds and combinations disclosed herein include cancer selected from lung cancer (NSCLC and SCLC), cancer of the head or neck, ovarian cancer, colon cancer, rectal cancer, prostate cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, or a combination of one or more of the foregoing cancers.

In some embodiments, the compounds and combinations disclosed herein are useful for the treatment of cancers, including Spitz melanoma, perineural invasion, pulmonary large cell neuroendocrine carcinoma, uterine carcinoma, juvenile breast cancer, nasopharyngeal carcinoma, adenoid cystic cancer, meduallary thyroid cancer, salivary cancer, congenital infantile fibrosarcoma, mesoblastic nephroma, esophageal cancer (squamous), diffuse large B-cell lymphoma, papillary thyroid cancer, and mammary analogue secretory carcinoma.

In some embodiments, the compounds and combinations disclosed herein may be used in combination with one or more additional anti-cancer agents which are described below. When a combination therapy is used, the one or more additional anti-cancer agents may be administered sequentially or simultaneously with the compound disclosed herein. In some embodiments, the additional anti-cancer agent is administered to a mammal (e.g., a human) prior to administration of the compound disclosed herein. In some embodiments, the additional anti-cancer agent is administered to the mammal after administration of the compound disclosed herein. In some embodiments, the additional anti-cancer agent is administered to the mammal (e.g., a human) simultaneously with the administration of compounds disclosed herein, or a pharmaceutically acceptable salt thereof.

Some embodiments also relate to a pharmaceutical composition for the treatment of abnormal cell growth in a mammal, including a human, which comprises an amount of one or more compounds disclosed herein, or a pharmaceutically acceptable salt thereof, including hydrates, solvates and polymorphs of said compound or pharmaceutically acceptable salts thereof, in combination at least one PD-1 inhibitor or anti-PD-L1 agent or a combination thereof, and with one or more (preferably one to three) anti-cancer agents selected from the group consisting of anti-angiogenesis agents and signal transduction inhibitors and a pharmaceutically acceptable carrier, wherein the amounts of the active agent and the combination anti-cancer agents when taken as a whole is therapeutically effective for treating said abnormal cell growth.

In some embodiments, the anti-cancer agent used in conjunction with one or more compounds disclosed herein, or a pharmaceutically acceptable salt thereof, and pharmaceutical compositions described herein is an anti-angiogenesis agent (e.g., an agent that stops tumors from developing new blood vessels). Examples of anti-angiogenesis agents include for example VEGF inhibitors, VEGFR inhibitors, TIE-2 inhibitors, PDGFR inhibitors, angiopoetin inhibitors, PKC beta inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent®), bevacizumab (Avastin®), axitinib (AG 13736), SU 14813 (Pfizer), and AG 13958 (Pfizer).

Additional anti-angiogenesis agents include vatalanib (CGP 79787), Sorafenib (Nexavar®), pegaptanib octasodium (Macugen®), vandetanib (Zactima®), PF-0337210 (Pfizer), SU 14843 (Pfizer), AZD 2171 (AstraZeneca), ranibizumab (Lucentis®), Neovastat® (AE 941), tetrathiomolybdata (Coprexa®), AMG 706 (Amgen), VEGF Trap (AVE 0005), CEP 7055 (Sanofi-Aventis), XL 880 (Exelixis), telatinib (BAY 57-9352), and CP-868,596 (Pfizer).

Other anti-angiogenesis agents include enzastaurin (LY 317615), midostaurin (CGP 41251), perifosine (KRX 0401), teprenone (Selbex®) and UCN 01 (Kyowa Hakko).

Other examples of anti-angiogenesis agents which can be used in conjunction with the compounds and combinations disclosed herein herein include celecoxib (Celebrex®), parecoxib (Dynastat®), deracoxib (SC 59046), lumiracoxib (Preige®), valdecoxib (Bextra®), rofecoxib (Vioxx®), iguratimod (Careram®), IP 751 (Invedus), SC-58125 (Pharmacia) and etoricoxib (Arcoxia®).

Other anti-angiogenesis agents include exisulind (Aptosyn®), salsalate (Amigesic®), diflunisal (Dolobid®), ibuprofen (Motrin®), ketoprofen (Orudis®) nabumetone (Relafen®), piroxicam (Feldene®), naproxen (Aleve®, Naprosyn®) diclofenac (Voltaren®), indomethacin (Indocin®), sulindac (Clinoril®), tolmetin (Tolectin®), etodolac (Lodine®), ketorolac (Toradol®), and oxaprozin (Daypro®).

Other anti-angiogenesis agents include ABT 510 (Abbott), apratastat (TMI 005), AZD 8955 (AstraZeneca), incyclinide (Metastat®), and PCK 3145 (Procyon).

Other anti-angiogenesis agents include acitretin (Neotigason®), plitidepsin (Aplidine®), cilengtide (EMD 121974), combretastatin A4 (CA4P), fenretinide (4 HPR), halofuginone (Tempostatin®), Panzem® (2-methoxyestradiol), PF-03446962 (Pfizer), rebimastat (BMS 275291), catumaxomab (Removab®), lenalidomide (Revlimid®) squalamine (EVIZON®), thalidomide (Thalomid®), Ukrain® (NSC 631570), Vitaxin® (MEDI 522), and zoledronic acid (Zometa®).

In some embodiments, the anti-cancer agent is a so called signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, ALK inhibitors, ROS1 inhibitors, TrkA inhibitors, TrkB inhibitors, TrkC inhibitors, farnesyl protein transferase inhibitors, EGF inhibitor, ErbB-1 (EGFR), ErbB-2, pan erb, IGF1R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WNT pathway and so called multi-targeted kinase inhibitors.

Preferred signal transduction inhibitors include gefitinib (Iressa®), cetuximab (Erbitux®), erlotinib (Tarceva®), trastuzumab (Herceptin®), sunitinib (Sutent®) imatinib (Gleevec®), and PD325901 (Pfizer).

Additional examples of signal transduction inhibitors which may be used in conjunction with one or more compounds of Disclosed herein and pharmaceutical compositions described herein include BMS 214662 (Bristol-Myers Squibb), lonafarnib (Sarasar®), pelitrexol (AG 2037), matuzumab (EMD 7200), nimotuzumab (TheraCIM h-R3®), panitumumab (Vectibix®), Vandetanib (Zactima®), pazopanib (SB 786034), ALT 110 (Alteris Therapeutics), BIBW 2992 (Boehringer Ingelheim), and Cervene® (TP 38).

Other examples of signal transduction inhibitor include PF-2341066 (Pfizer), PF-299804 (Pfizer), canertinib (CI 1033), pertuzumab (Omnitarg®), Lapatinib (Tycerb®), pelitinib (EKB 569), miltefosine (Miltefosin®), BMS 599626 (Bristol-Myers Squibb), Lapuleucel-T (Neuvenge®), NeuVax® (E75 cancer vaccine), Osidem® (IDM 1), mubritinib (TAK-165), CP-724,714 (Pfizer), panitumumab (Vectibix®), lapatinib (Tycerb®), PF-299804 (Pfizer), pelitinib (EKB 569), and pertuzumab (Omnitarg®).

Other examples of signal transduction inhibitors include ARRY 142886 (Array Biopharm), everolimus (Certican®), zotarolimus (Endeavor®), temsirolimus (Torisel®), AP 23573 (ARIAD), and VX 680 (Vertex).

Additionally, other signal transduction inhibitors include XL 647 (Exelixis), sorafenib (Nexavar®), LE-AON (Georgetown University), and GI-4000 (GlobeImmune).

Other signal transduction inhibitors include ABT 751 (Abbott), alvocidib (flavopiridol), BMS 387032 (Bristol Myers), EM 1421 (Erimos), indisulam (E 7070), seliciclib (CYC 200), BIO 112 (One Bio), BMS 387032 (Bristol-Myers Squibb), PD 0332991 (Pfizer), AG 024322 (Pfizer), entrectinib, RXDX-105 (Ignyta), LOXO-101 (Loxo Oncology), crizotinib, and ceritinib.

In some embodiments, the compounds disclosed herein, or a pharmaceutically acceptable salt thereof, are used together with classical antineoplastic agents. Classical antineoplastic agents include but are not limited to hormonal modulators such as hormonal, anti-hormonal, androgen agonist, androgen antagonist and anti-estrogen therapeutic agents, histone deacetylase (HDAC) inhibitors, gene silencing agents or gene activating agents, ribonucleases, proteosomics, Topoisomerase I inhibitors, Camptothecin derivatives, Topoisomerase II inhibitors, alkylating agents, antimetabolites, poly(ADP-ribose) polymerase-1 (PARP-1) inhibitor, microtubulin inhibitors, antibiotics, plant derived spindle inhibitors, platinum-coordinated compounds, gene therapeutic agents, antisense oligonucleotides, vascular targeting agents (VTAs), and statins.

Examples of classical antineoplastic agents used in combination therapy with one or more compounds disclosed herein, or a pharmaceutically acceptable salt thereof, optionally with one or more other agents include, but are not limited to, glucocorticoids, such as dexamethasone, prednisone, prednisolone, methylprednisolone, hydrocortisone, and progestins such as medroxyprogesterone, megestrol acetate (Megace), mifepristone (RU-486), Selective Estrogen Receptor Modulators (SERMs; such as tamoxifen, raloxifene, lasofoxifene, afimoxifene, arzoxifene, bazedoxifene, fispemifene, ormeloxifene, ospemifene, tesmilifene, toremifene, trilostane and CHF 4227 (Cheisi), Selective Estrogen-Receptor Downregulators (SERD's; such as fulvestrant), exemestane (Aromasin), anastrozole (Arimidex), atamestane, fadrozole, letrozole (Femara), gonadotropin-releasing hormone (GnRH; also commonly referred to as luteinizing hormone-releasing hormone [LHRH]) agonists such as buserelin (Suprefact), goserelin (Zoladex), leuprorelin (Lupron), and triptorelin (Trelstar), abarelix (Plenaxis), bicalutamide (Casodex), cyproterone, flutamide (Eulexin), megestrol, nilutamide (Nilandron), and osaterone, dutasteride, epristeride, finasteride, Serenoa repens, PHL 00801, abarelix, goserelin, leuprorelin, triptorelin, bicalutamide, tamoxifen, exemestane, anastrozole, fadrozole, formestane, letrozole, and combinations thereof.

Other examples of classical antineoplastic agents used in combination with compounds disclosed herein, or a pharmaceutically acceptable salt thereof, include, but are not limited to, suberolanilide hydroxamic acid (SAHA, Merck Inc./Aton Pharmaceuticals), depsipeptide (FR901228 or FK228), G2M-777, MS-275, pivaloyloxymethyl butyrate and PXD-101; Onconase (ranpirnase), PS-341 (MLN-341), Velcade (bortezomib), 9-aminocamptothecin, belotecan, BN-80915 (Roche), camptothecin, diflomotecan, edotecarin, exatecan (Daiichi), gimatecan, 10-hydroxycamptothecin, irinotecan HCl (Camptosar), lurtotecan, Orathecin (rubitecan, Supergen), SN-38, topotecan, camptothecin, 10-hydroxycamptothecin, 9-aminocamptothecin, irinotecan, SN-38, edotecarin, topotecan, aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide, AMD-473, altretamine, AP-5280, apaziquone, brostallicin, bendamustine, RXDX-107 (Ignyta), busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin, Eloxatin (oxaliplatin, Sanofi), streptozocin, satrplatin, and combinations thereof.

In some embodiments, the compounds disclosed herein, or a pharmaceutically acceptable salt thereof, are used together with dihydrofolate reductase inhibitors (such as methotrexate and NeuTrexin (trimetresate glucuronate), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda®), cytosine arabinoside, Gemzar® (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, N-(5-[N-(3,4-dihydro-2-methyl-4-oxoquinazolin-6-ylmethyl)-N-methylamino]-2-thenoyl)-L-glutamic acid, AG-014699 (Pfizer Inc.), ABT-472 (Abbott Laboratories), INO-1001 (Inotek Pharmaceuticals), KU-0687 (KuDOS Pharmaceuticals) and GPI 18180 (Guilford Pharm Inc.) and combinations thereof.

Other examples of classical antineoplastic cytotoxic agents used in combination therapy with one or more compounds disclosed herein, or a pharmaceutically acceptable salt thereof, optionally with one or more other agents include, but are not limited to, Abraxane (Abraxis BioScience, Inc.), Batabulin (Amgen), EPO 906 (Novartis), Vinflunine (Bristol-Myers Squibb Company), actinomycin D, bleomycin, mitomycin C, neocarzinostatin (Zinostatin), vinblastine, vincristine, vindesine, vinorelbine (Navelbine), docetaxel (Taxotere), Ortataxel, paclitaxel (including Taxoprexin a DHA/paciltaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta®), DMXAA (Antisoma), ibandronic acid, L-asparaginase, pegaspargase (Oncaspar®), Efaproxiral (Efaproxyn®—radiation therapy), bexarotene (Targretin®), Tesmilifene (DPPE—enhances efficacy of cytotoxics), Theratope® (Biomira), Tretinoin (Vesanoid®), tirapazamine (Trizaone®), motexafin gadolinium (Xcytrin®) Cotara® (mAb), and NBI-3001 (Protox Therapeutics), polyglutamate-paclitaxel (Xyotax®) and combinations thereof.

Further examples of classical antineoplastic agents used in combination therapy with one or more compounds disclosed herein, or a pharmaceutically acceptable salt thereof, optionally with one or more other agents include, but are not limited to, as Advexin (ING 201), TNFerade (GeneVec, one or more compounds which express TNFalpha in response to radiotherapy), RB94 (Baylor College of Medicine), Genasense (Oblimersen, Genta), Combretastatin A4P (CA4P), Oxi-4503, AVE-8062, ZD-6126, TZT-1027, Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin, Niacin (Advicor, Kos Pharmaceuticals), Caduet, Lipitor, torcetrapib, and combinations thereof.

Some embodiments relate to a method for the treatment of breast cancer in a human in need of such treatment, comprising administering to said human an amount of one or more compounds or combinations disclosed herein in combination with one or more (preferably one to three) anti-cancer agents selected from the group consisting of trastuzumab, tamoxifen, docetaxel, paclitaxel, capecitabine, gemcitabine, vinorelbine, exemestane, letrozole and anastrozole.

Some embodiments provide a method of treating colorectal cancer in a mammal, such as a human, in need of such treatment, by administering an amount of one or more compounds or combinations disclosed herein in combination with one or more (preferably one to three) anti-cancer agents. Examples of particular anti-cancer agents include those typically used in adjuvant chemotherapy, such as FOLFOX, a combination of 5-fluorouracil (5-FU) or capecitabine (Xeloda), leucovorin and oxaliplatin (Eloxatin). Further examples of particular anti-cancer agents include those typically used in chemotherapy for metastatic disease, such as FOLFOX or FOLFOX in combination with bevacizumab (Avastin); and FOLFIRI, a combination of 5-FU or capecitabine, leucovorin and irinotecan (Camptosar). Further examples include 17-DMAG, ABX-EFR, AMG-706, AMT-2003, ANX-510 (CoFactor), aplidine (plitidepsin, Aplidin), Aroplatin, axitinib (AG-13736), AZD-0530, AZD-2171, bacillus Calmette-Guerin (BCG), bevacizumab (Avastin), BIO-117, BIO-145, BMS-184476, BMS-275183, BMS-528664, bortezomib (Velcade), C-1311 (Symadex), cantuzumab mertansine, capecitabine (Xeloda), cetuximab (Erbitux), clofarabine (Clofarex), CMD-193, combretastatin, Cotara, CT-2106, CV-247, decitabine (Dacogen), E-7070, E-7820, edotecarin, EMD-273066, enzastaurin (LY-317615) epothilone B (EPO-906), erlotinib (Tarceva), flavopyridol, GCAN-101, gefitinib (Iressa), huA33, huC242-DM4, imatinib (Gleevec), indisulam, ING-1, irinotecan (CPT-11, Camptosar) ISIS 2503, ixabepilone, lapatinib (Tykerb), mapatumumab (HGS-ETR1), MBT-0206, MEDI-522 (Abregrin), Mitomycin, MK-0457 (VX-680), MLN-8054, NB-1011, NGR-TNF, NV-1020, oblimersen (Genasense, G3139), OncoVex, ONYX 015 (CI-1042), oxaliplatin (Eloxatin), panitumumab (ABX-EGF, Vectibix), pelitinib (EKB-569), pemetrexed (Alimta), PD-325901, PF-0337210, PF-2341066, RAD-001 (Everolimus), RAV-12, Resveratrol, Rexin-G, S-1 (TS-1), seliciclib, SN-38 liposome, Sodium stibogluconate (SSG), sorafenib (Nexavar), SU-14813, sunitinib (Sutent), temsirolimus (CCI 779), tetrathiomolybdate, thalomide, TLK-286 (Telcyta), topotecan (Hycamtin), trabectedin (Yondelis), vatalanib (PTK-787), vorinostat (SAHA, Zolinza), WX-UK1, and ZYC300, wherein the amounts of the active agent together with the amounts of the combination anticancer agents are effective in treating colorectal cancer.

Some embodiments provide methods for the treatment of renal cell carcinoma in a human in need of such treatment, comprising administering to said human an amount of one or more compounds or combinations disclosed herein in combination with one or more (preferably one to three) anti-cancer agents selected from the group consisting of capecitabine (Xeloda), interferon alpha, interleukin-2, bevacizumab (Avastin), gemcitabine (Gemzar), thalidomide, cetuximab (Erbitux), vatalanib (PTK-787), Sutent, AG-13736, SU-11248, Tarceva, Iressa, Lapatinib and Gleevec, wherein the amounts of the active agent together with the amounts of the combination anticancer agents is effective in treating renal cell carcinoma.

Some embodiments provide methods for the treatment of melanoma in a human in need of such treatment, comprising administering to said human an amount of one or more compounds or combinations disclosed herein in combination with one or more (preferably one to three) anti-cancer agents selected from the group consisting of interferon alpha, interleukin-2, temozolomide (Temodar), docetaxel (Taxotere), paclitaxel, Dacarbazine (DTIC), carmustine (also known as BCNU), Cisplatin, vinblastine, tamoxifen, PD-325,901, Axitinib, bevacizumab (Avastin), thalidomide, sorafanib, vatalanib (PTK-787), Sutent, CpG-7909, AG-13736, Iressa, Lapatinib and Gleevec, wherein the amounts of the compound disclosed herein, or a pharmaceutically acceptable salt thereof, together with the amounts of the combination anticancer agents is effective in treating melanoma.

Some embodiments provide methods for the treatment of lung cancer in a human in need of such treatment, comprising administering to said human an amount of one or more compounds or combinations disclosed herein in combination with one or more (preferably one to three) anti-cancer agents selected from the group consisting of capecitabine (Xeloda), bevacizumab (Avastin), gemcitabine (Gemzar), docetaxel (Taxotere), paclitaxel, premetrexed disodium (Alimta), Tarceva, Iressa, Vinorelbine, Irinotecan, Etoposide, Vinblastine, and Paraplatin (carboplatin), wherein the amounts of the active agent together with the amounts of the combination anticancer agents is effective in treating lung cancer.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc., are presented merely for ease of reading the specification and claims. The use of headings in the specification or claims does not require the steps or elements be performed in alphabetical or numerical order or the order in which they are presented.

The following are intended to comprise examples of a number of embodiments and are intended to be illustrative and not limiting.

### EXAMPLES

The following abbreviations and definitions may be used in the examples that follow are intended to have the following meanings: “BLI” means bioluminescent imaging, “HEPES” means 4-(2-hydroxyethyl)-1-piperazineethanesulfonic acid, “IP” means injection of a substance into the peritoneum, “PBS” means phosphate buffered saline, “PEG” means polyethylene glycol, “HEPES” means 4-(2-hydroxyethyl)-1-piperazineethanesulfonic acid, and “PO” means per os or dosing by mouth.

### Example 1

Combinations comprising (a) N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide and an anti-PD-1 agent, and (b) N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide and an anti-PD-L1 agent are tested in mice implanted with cells from one of four tumors: (1) CT-26 (colon); (2) MC38 (colon); (3) B16/F10 (melanoma), and (4) LLC (lung).

The anti-PD-L1 and anti-PD-1 agents are prepared as clear, colorless stock solutions. The dosing solutions are prepared by diluting 1.5 mL stock solutions of each agent with 7.9 mL phosphate buffered saline solution to afford 1 mg per mL dosing solutions with a pH value of 7. The dosing formulations are prepared weekly, and stored protected from the light at 4° C. when not in use.

The mice implanted with each of the four tumor types are sorted into one of the seven study groups below based on body weight. The animals are distributed to ensure that the mean body weight for all groups is within 10% of the overall mean for the study population. Treatment is begun on Day 3, prior to the tumors being palpable. All animals are dosed 0.2 mL per 20 g of body weight.

Group 1: Vehicle Control (50% PEG400; 50% oleic acid) PO, once daily for 15 days (Days 3-17)

Group 2: N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, 30 mg/kg PO, once daily for 15 days (Days 3-17)

Group 3: N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, 10 mg/kg PO, once daily for 15 days (Days 3-17)

Group 4: N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, 1 mg/kg PO, once daily for 15 days (Days 3-17)

Group 5: anti-PD-L1 agent, 10 mg/kg IP, every third day for 2 treatments, then a 3 day rest for 2.5 weeks (Days 3, 6, 10, 13, 17)

Group 6: anti-PD-L1, 10 mg/kg IP, every third day for 2 treatments, then a 3 day rest for 2.5 weeks (Days 3, 6, 10, 13, 17), plus N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, 0.1 to 50 mg/kg PO, once daily for 15 days (Days 3-17)

Group 7: anti-PD-1, 10 mg/kg IP, every third day for 2 treatments, then a 3 day rest for 2.5 weeks (Days 3, 6, 10, 13, 17), plus N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, 0.1 to 50 mg/kg PO, once daily for 15 days (Days 3-17)

At 2 hours following the final dose for Groups 1-7 on Day 17, all animals are euthanized for blood, spleen, and tumor collection. Dosing times are staggered to allow for sample collection. All animals are euthanized via over-exposure to carbon dioxide. Whole blood is collected via cardiac puncture. Following collection, all whole blood is added to serum containers and allowed to clot at ambient temperature for approximately 30 minutes. The samples are then centrifuged in a refrigerated (4° C.) centrifuge at 9300rcf for 5 minutes to generate serum. All serum samples are then frozen and stored at −80° C. until they are analyzed.

Testing in this experiment is generally carried out adhering to the general principles established by the groups of Schabel, Skipper, Griswold, Corbett, Leopold, Ross and the NCI. Tumor measurements are recorded daily. Tumor burden (mm3) is estimated from caliper measurements by the formula for the volume of a prolate ellipsoid assuming unit density as: Tumor burden (mm3)=(L×W2)/2, where L and W are the respective orthogonal tumor length and width measurements (mm). Animals with tumors in excess of 2000 mm3 are euthanized, as are those found in obvious distress or in a moribund condition.

The primary endpoints used to evaluate efficacy are: Day 14 treatment/control (T/C) by caliper measurement and Day 14 T/C by whole body bioluminescence imaging (BLI). % T/C is defined as the median tumor burden of the Treated Group divided by the median tumor mass of the Control Group×100. Tumor growth delay is inappropriate as an endpoint because of the scheduled termination of the study on Day 17 which precludes growth delays longer than 1.4 days.

Significantly reduced tumor growth is expected to be observed in animals in groups 6 and 7 for each of the four tumor types compared to tumor growth in animals in groups 1 to 5.

### Example 2

In vivo efficacy of a combination comprising N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide and an anti-PD-1 antibody were tested in mice implanted with cells from CT-26 colon cancer.

Particularly, BALB/c female mice between 6-7 weeks of age were ordered from Charles River. Animals were allowed to acclimate 3 days prior to the start of the study (e.g., cell implantation). All animals were housed in irradiated, individual HEPA ventilated cages (Innocage® IVC, Innovive USA) on a 12-hour light-dark cycle at 68-79° F. and 30-70% humidity. Animals were provided with irradiated chow (Teklad 2920X) and acidified drinking water (Innovive) ad libitum as per the animal care and use protocol established at Explora Biolabs. Animals were identified by uniquely numbered ear-tags.

CT-26 cells were cultured and suspended in sterile PBS (Gibco) at a concentration of 2.5 million cells/mL. Cells were implanted under the skin on the right flank of each mouse on day 0. The implantation volume was 200 μL/mouse. Tumors grew until the mean tumor volume reached approximately 125 mm³. On day 8 mice were randomized into treatment groups (a) to (e) as described below) (10 mice/group) and treatment began.

Animals were treated by administering with (a) vehicle (1:1 Oleic Acid (Fisher) and PEG400 (Sigma)), (b) Iso type antibody for α-PD-1 (clone 2A3, BioXcell), (c) α-PD-1 antibody (clone PMP1-14, BioXcell), (d) compound N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, or (e) a combination of the compound N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide and the antibody.

The vehicle and the compound N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide were dosed (30 mg/kg) orally on a daily schedule (p.o. q.d.). Antibody was dosed (10 mg/kg) intraperitoneally (i.p.) on a twice a week schedule. The final tumor and body weight measurements were taken on day 27. Two mice in treatment group (e) died on days 8 and 20, respectively. All other mice remained on study until its completion.

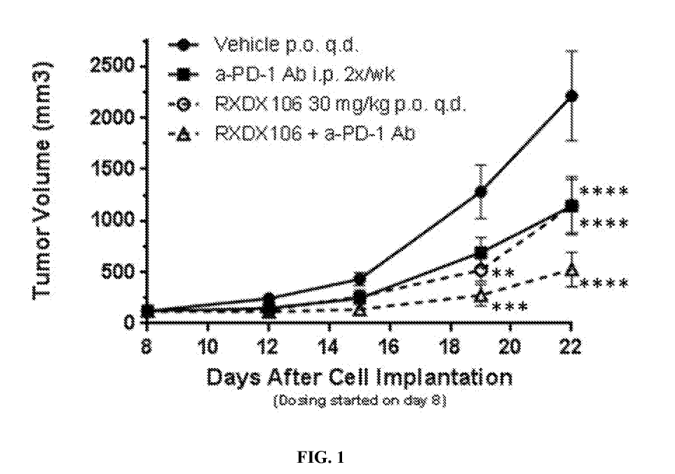

Tumors measurements and body weights were collected twice a week over the duration of the study. Tumor growth was assessed by caliper and tumor volumes were calculated using the equation; Volume (mm³)=[length (mm)×width (mm)×width (mm)]/2. Animals with tumor volumes>2,000 mm³ were removed from the study. Body weights were collected using a laboratory scale. Toxicity was evaluated based on body weight loss. Animals losing>15% of their body weight were removed from the study. FIG. 1 shows the results of tumor measurments for treatment groups (a) solid circle, (c) square, (d) open circle and (e) triangle.

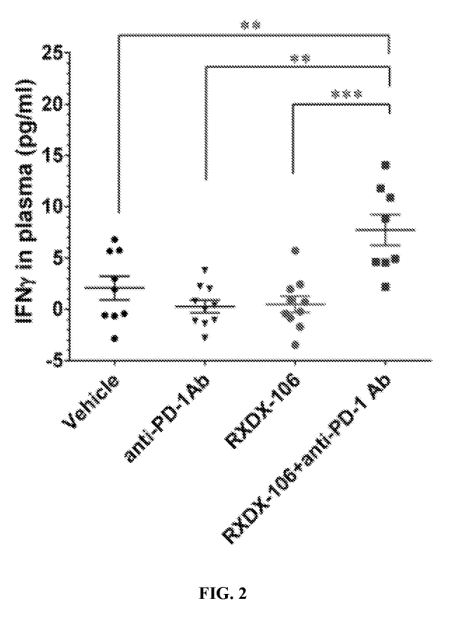

At end of study on day 22, blood sample was collected. Following collection, all whole blood is added to K2 EDTA coated tubes and stored on ice for approximately 30 minutes. The samples are centrifuged in a refridgerated centrifuge (4° C.) at 1000×g for 20 minutes. Plasma is collected and frozen at −80° C. until they are analyzed. Interferon gamma levels were measured by ELISA using a mouse interferon gamma kit purchased from R&D Systems (Catalog #DY485). FIG. 2 shows the interferon gamma levels measured from treatment groups (a), (c), (d) and (e), respectively.

Data analysis was performed using Graphpad Prism. Data represent mean+/−standard error mean. Statistical analysis was performed using a student's T test.

As shown in FIG. 1, treatment group (a) treated with vehicle only had greater tumor volumns than the remaining groups consistently throughout the study; Treatment group (e) treated with the combination of the compound and anti-PD-1 antibody consistently had smaller tumor volumns than the ramining groups consistently throughout the study. These results clearly show increased in vivo efficacy in inhibiting tumor growth brought by comibining the anti-PD-1 antibody with the kinase inhibitor compound N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide. This synergistic anti-tumor effect may be explained by a significant increase in IFNγ secretion in mice treated with the combination, as shown in FIG. 2.

1. A method of treating cancer in a subject, the method comprising administering to said subject a therapeutically effective amount of a combination comprising an inhibitor of Tyro3, Axl, Mer, or c-Met; and an anti-PD-1 agent or an anti-PD-L1 agent.

2. A method of treating, ameliorating the symptoms of, delaying the onset of, or delaying the progression of cancer in a subject, the method comprising:
determining whether modulation of Tyro3, Axl, Mer, or c-Met activity is defective in a cell population of said subject, and if said modulation of Tyro3, Axl, Mer, or c-Met activity is defective, and
administering a combination to said subject, the combination comprising an inhibitor of Tyro3, Axl, Mer, or c-Met; and an anti-PD-1 agent or an anti-PD-L1 agent thereby treating, ameliorating the symptoms of, delaying the onset of, or delaying the progression of cancer.

3-8. (canceled)

9. The metohd of claim 1, wherein said combination comprises N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, or a pharmaceutically acceptable salt thereof, and an anti-PD-1 agent or an anti-PD-L1 agent.

10. The method according to claim 1, wherein said anti-PD-1 agent or anti-PD-L1 agent is a monoclonal antibody.

11. The method of claim 10, wherein said monoclonal antibody is a fully human monoclonal antibody.

12. The method of claim 10, wherein said anti-PD-1 agent or anti-PD-L1 agent is selected from the group consisting of nivolumab, pembrolizumab, atezolizumab, avelumab, and durvalumab.

13-17. (canceled)

18. The method of claim 1, wherein said inhibitor of Tyro3, Axl, Mer, or c-Met and said anti-PD-1 agent or anti-PD-L1 agent are administered to said subject simultaneously.

19. The method of claim 1, wherein said inhibitor of Tyro3, Axl, Mer, or c-Met and said anti-PD-1 agent or anti-PD-L1 agent are administered to said subject sequentially.

20. The method of claim 1, wherein said inhibitor of Tyro3, Axl, Mer, or c-Met is administered to said subject orally.

21. The method of claim 1, wherein said anti-PD-1 agent or anti-PD-L1 agent is administered to said subject intravenously.

22. (canceled)

23. The method of claim 1, wherein said anti-PD-1 agent or anti-PD-L1 agent is administered to said subject every 3 weeks.

24. The method of claim 1, wherein said anti-PD-1 agent or anti-PD-L1 agent is administered to said subject in four doses every 3 weeks.

25. The method of claim 1, wherein said anti-PD-1 agent or anti-PD-L1 agent is administered to said subject in a dose of 3 mg per kilogram of the weight of said subject.

26-27. (canceled)

28. The method of claim 1, wherein said anti-PD-1 agent or anti-PD-L1 agent is administered to said subject in a dose of 3 mg per kilogram of the weight of said subject every 3 weeks for a total of 4 doses.

29. The method of claim 1, wherein said inhibitor of Tyro3, Axl, Mer, or c-Met is administered to said subject at least once per day.

30. The method of claim 1, wherein said inhibitor of Tyro3, Axl, Mer, or c-Met is administered to said subject in a dose of about 0.1 mg per kilogram of patient weight to about 1000 mg per kilogram of patient weight.

31. The method of claim 1, wherein said inhibitor of Tyro3, Axl, Mer, or c-Met is N-[4-[(6,7-dimethoxy-4-quinolinyl)oxy]-3-fluorophenyl]-3-(4-fluorophenyl)-1,2,3,4-tetrahydro-1-(1-methylethyl)-2,4-dioxo-5-pyrimidinecarboxamide, or a pharmaceutically acceptable salt thereof.

32. The method of claim 1, wherein one or more cancer cells in said subject is determined to possess at least one molecular alteration in one or more of Tyro3, Axl, Mer, or c-Met prior to administration of said inhibitor of Tyro3, Axl, Mer, or c-Met.

33-36. (canceled)

37. The method of claim 1, wherein said cancer is selected from the group consisting of heart sarcoma, lung cancer, small cell lung cancer (SCLC), non-small cell lung cancer (NSCLC), bronchogenic carcinoma, alveolar (bronchiolar) carcinoma, bronchial adenoma, sarcoma, lymphoma, chondromatous hamartoma, mesothelioma; gastrointestinal cancer, genitourinary tract cancer, liver cancer, bone cancer, nervous system cancer, reproductive system cancer, hematologic system cancer, oral cavity cancer, skin cancer, cancer of adrenal glands, neuroblastoma, retroperitoneum cancer, peritoneum cancer, eye cancer, intraoculat melanoma, adnexa cancer, breast cancer, head cancer, neck cancer, anal region cancer, thyroid cancer, parathyroid cancer, secondary and unspecificed malignant neoplasm of lymph nodes, and secondary malignant neoplasma of respriatory and digestive systems.

38-40. (canceled)

41. A kit, comprising:
(a) a first composition comprising an inhibitor of Tyro3, Axl, Mer, or c-Met;
(b) a second composition comprising an anti-PD-1 agent or an anti-PD-L1 agent; and
(c) instructions for use of said first composition and said second composition in the treatment of cancer in a subject.

42.-82. (canceled)
